(19) United States
(12) Patent Application Publication
(10) Pub. No.: US 2010/0035823 A1
(43) Pub. Date: Feb. 11, 2010

(54) LIGANDS OF SH3 DOMAINS

(75) Inventor: David S. Lawrence, Chapel Hill, NC (US)

Correspondence Address: AMSTER, ROTHSTEIN & EBENSTEIN LLP, 90 PARK AVENUE, NEW YORK, NY, 10016, US

(73) Assignee: ALBERT EINSTEIN COLLEGE OF MEDICINE OF YESHIVA UNIVERSITY, Bronx, NY (US)

(21) Appl. No.: 11/989,718

(22) PCT Filed: Aug. 8, 2006
(86) PCT No.: PCT/US2006/030799

§ 371 Date: Oct. 13, 2009

Related U.S. Application Data
(60) Provisional application No. 60/707,799, filed on Aug. 11, 2005.

(51) Int. Cl. A61K 38/08; C07K 7/06; C40B 40/10; C40B 30/04

(52) U.S. Cl. 514/16; 530/329; 530/328; 506/18; 506/9

(57) ABSTRACT

Provided are compounds that bind to SH3 domains. Also provided are combinatorial libraries for discovering such compounds. Methods of identifying a compound that binds to an SH3 domain are also provided. Methods of inhibiting an activity of a protein comprising an SH3 domain are additionally provided. Additionally provided are methods of inhibiting the activity of a protein comprising an SH3 domain and methods of treating a mammal having a deleterious condition that is mediated by a protein comprising an SH3 domain. The use of a compound that binds to the SH3 domain of a protein kinase in the manufacture of a medicament for the treatment of a deleterious condition in a mammal that is dependent on a protein kinase for induction or severity are also provided.

### CROSS-REFERENCE TO RELATED APPLICATION

This application claims the benefit of U.S. Provisional Application No. 60/707,799, filed Aug. 11, 2005.

### STATEMENT REGARDING FEDERALLY SPONSORED RESEARCH OR DEVELOPMENT

The U.S. Government has a paid-up license in this invention and the right in limited circumstances to require the patent owner to license others on reasonable terms as provided for by the terms of Grant No. CA79954 awarded by The National Institutes of Health.

### BACKGROUND OF THE INVENTION

(1) Field of the Invention

The present invention generally relates to ligands of protein domains. More specifically, the invention is directed to ligands and inhibitors of SH3 domains and proteins having SH3 domains.

(2) Description of the Related Art

### REFERENCES CITED

Badour, K., Zhang, J., Shi, F., Leng, Y., Collins, M., and Siminovitch, K. A. (2004). Fyn and PTP-PEST-mediated regulation of Wiskott-Aldrich syndrome protein (WASp) tyrosine phosphorylation is required for coupling T cell antigen receptor engagement to WASp effector function and T cell activation. J Exp Med 199, 99-112.

Banin, S., Truong, O., Katz, D. R., Waterfield, M. D., Brickell, P. M., and Gout, I. (1996). Wiskott-Aldrich syndrome protein (WASp) is a binding partner for c-Src family protein-tyrosine kinases. Curr Biol 6, 981-988.

Boggon, T. J., and Eck, M. J. (2004). Structure and regulation of Src family kinases. Oncogene 23, 7918-7927.

Borreguero, J. M., Dokholyan, N. V., Buldyrev, S. V., Shakinovich, E. I., and Stanley, H. E. (2002). Thermodynamics and Folding Kinetics Analysis of the SH3 Domain from Discrete Molecular Dynamics. J Mol Biol 318, 863-876.

Cesareni, G., Panni, S., Nardelli, G., and Castagnoli, L. (2002). Can we infer peptide recognition specificity mediated by SH3 domains? FEBS Lett 513, 38-44.

Feng, S., Chen, J. K., Yu, H., Simon, J. A., and Schreiber, S. L. (1994). Two binding orientations for peptides to the Src SH3 domain: development of a general model for SH3-ligand interactions. Science 266, 1241-1247.

Feng, S., Kapoor, T. M., Shirai, F., Combs, A. P., and Schreiber, S. L. (1996). Molecular basis for the binding of SH3 ligands with non-peptide elements identified by combinatorial synthesis. Chem Biol 3, 661-670.

Gmeiner, W. H., and Horita, D. A. (2001). Implications of SH3 domain structure and dynamics for protein regulation and drug design. Cell Biochem Biophys 35, 127-140.

Gu, J., and Gu, X. (2003). Natural history and functional divergence of protein tyrosine kinases. Gene 317, 49-57.

Hanafusa, H. (1995). Affinity and specificity requirements for the first Src homology 3 domain of the Crk proteins. Embo J 14, 2191-2198.

Kieber-Emmons et al. (1997). Curr Opin Biotechnol 8, 435-441.

Knudsen, B. S., Zheng, J., Feller, S. M., Mayer, J. P., Burrell, S. K., Cowburn, D., and

Lee, T. R., and Lawrence, D. S. (1999). Acquisition of high-affinity, SH2-targeted ligands via a spatially focused library. J Med Chem 42, 784-787.

Lee, J. H., Nandy, S. K., and Lawrence, D. S. (2004). A highly potent and selective PKCalpha inhibitor generated via combinatorial modification of a peptide scaffold. J Am Chem Soc 126, 3394-3395.

Mayer, B. J. (2001). SH3 domains: complexity in moderation. J Cell Sci 114, 1253-1263.

Morley, A. D. (2000). Optimisation and synthesis of libraries derived from phenolic amino acid scaffolds. Tetrahedron Letters 41, 7405-7408.

Musacchio, A. (2002). How SH3 domains recognize proline. Adv Protein Chem 61, 211-268.

Nguyen, J. T., Turck, C. W., Cohen, F. E., Zuckermann, R. N., and Lim, W. A. (1998). Exploiting the basis of proline recognition by SH3 and WW domains: design of N-substituted inhibitors. Science 282, 2088-2092.

Nguyen, J. T., Porter, M., Amoui, M., Miller, W. T., Zuckermann, R. N., and Lim, W. A. (2000). Improving SH3 domain ligand selectivity using a non-natural scaffold. Chem Biol 7, 463-473.

Palacios, E. H., and Weiss, A. (2004). Function of the Src-family kinases, Lck and Fyn, in T-cell development and activation. Oncogene 23, 7990-8000.

Pisabarro, M. T., and Serrano, L. (1996). Rational design of specific high-affinity peptide ligands for the Abl-SH3 domain. Biochemistry 35, 10634-10640.

Plaxco, K. W., Guijarro, J. I., Morton, C. J., Pitkeathly, M., Campbell, I. D., and Dobson, C. M. (1998). The folding kinetics and thermodynamics of the Fyn-SH3 domain. Biochemistry 37, 2529-2537.

Posern, G., Zheng, J., Knudsen, B. S., Kardinal, C., Muller, K. B., Voss, J., Shishido, T., Cowburn, D., Cheng, G., Wang, B., Kruli, G. D., Burrell, S. K., Jacobson, C. A., Lenz, D. M., Zamborelli, T. J., Adermami, K., Hanafusa, H., and Feller, S. M. (1998). Development of highly selective SH3 binding peptides for Crk and CRKL which disrupt Crk-complexes with DOCK180, SoS and C3G. Oncogene 16, 1903-1912.

Ripka et al. (1998). Curr Opin Chem Biol 2, 441-452.

Sanderson (1999). Med Res Rev 19, 179-197.

Schwende, H., Fitzke, E., Ambs, P., and Dieter, P. (1996). Differences in the state of differentiation of THP-1 cells induced by phorbol ester and 1,25-dihydroxyvitamin D3. J Leukoc Biol 59,-555-561.

Scott, M. P., Zappacosta, F., Kim, E. Y., Annan, R. S., and Miller, W. T. (2002). Identification of novel SH3 domain ligands for the Src family kinase Hck. Wiskott-Aldrich syndrome protein (WASP), WASP-interacting protein (WIP), and ELMOI. J Biol Chem 277, 28238-28246.

Shen, K., Keng, Y. F., Wu, L., Guo, X. L., Lawrence, D. S., and Zhang, Z. Y. (2001). Acquisition of a specific and potent PTPIB inhibitor from a novel combinatorial library and screening procedure. J Biol Chem 276, 47311-47319.

Yeh, R. H., Lee, T. R., and Lawrence, D. S. (2001). From consensus sequence peptide to high affinity ligand, a “library scan” strategy. J Biol Chem 276, 12235-12240.

Zarrinpar, A., Bhattacharyya, R. P., and Lim, W. A. (2003). The structure and function of proline recognition domains. Sci STKE 2003, RE8.

The Src homology 3 (SH3) domain is one of many protein recognition modules that play an essential role in the operation of signaling pathways (Musacchio, 2002; Mayer, 2001; Zarrinpar et al., 2003). Like all protein recognition units, the SH3 domain exhibits a marked preference for a particular consensus sequence of amino acid residues, namely a proline rich motif of the general form Pro-Xaa-Xaa-Pro (Cesareni et al., 2002). The molecular basis for the proline-based sequence preference has been resolved via the structural elucidation of several SH3 domain/peptide ligand complexes. The peptide ligand is bound as a type II polyproline helix. Side chains on every third residue of the helix are oriented in the same direction. Two of these side chains are positioned into hydrophobic pockets of the SH3 domain. In addition, other residues of the peptide ligand are also able to productively interact with the SH3 domain. One of the interesting aspects of the polyproline helix structure is that the ligand can associate with the binding pocket in an N-to-C orientation or in the opposite sense (Feng et al., 1994). The relative bound orientation is dictated by the presence of a positively charged residue on either the N-terminus of the peptide ligand (“Type I”) or at the corresponding C-terminus (“Type II”).

More than 400 different SH3 domains are encoded within the human genome. Consequently, even at this early stage in the analysis of the human proteome, it is not surprising that these domains have been implicated in a variety of normal and abnormal physiological processes (Gmeiner and Horita, 2001). At the biochemical level, SH3 domains have been shown to regulate enzymatic activity as well as promote the assembly of signaling complexes. For example, all nine members of the Src family of protein kinases contain the general structure (N-terminus)-SH3-SH2-SH1-(C-terminus) (Boggon and Eck, 2004). The SH3 domain not only serves as an intramolecular on/off switch that controls kinase activity, but also acts as a targeting moiety that directs Src kinase family members to the proper intracellular sites and substrates. SH3 domains are present in a variety of different proteins, including other tyrosine kinases, such as Abl and Crk, protein phosphatases, such as SHP-1, and adaptor proteins, such as Grb2 and Sos.

Many studies that seek to correlate the activity of a given signaling protein with cellular phenotype typically resort to the tools of molecular biology to create constitutively active, dominant negative, or other analogues of the corresponding wild type protein. Although this general strategy is a powerful one, deletion of the natural protein or expression of mutated analogues can result in complications arising from compensation by closely related proteins, inappropriate localization of the protein analogue, or unintended modulation of other signaling pathways. In contrast to genetically expressed protein analogues, inhibitors or synthetic ligands rapidly perturb the wild type protein, which is present in its natural state in terms of quantity, activity, and location. In this regard, SH3 domain-selective ligands are a much sought-after commodity. Unfortunately, the widespread presence of SH3 domains throughout the human proteome and their generally similar ligand recognition profiles renders this task a formidable one. In addition, several studies have demonstrated that conventional peptide ligands, consisting of standard amino acid (>10) residues, generally exhibit modest affinities (low μM) and low selectivities for their intended SH3 domain targets (Posem et al., 1998; Knudsen et al., 1995; Feng et al., 1996; Pisabarro, 1996). Indeed, protein interaction domains, in general, exhibit only moderately robust affinities for their endogenous ligands, a not too surprising observation given the fact that the protein-protein interactions that drive signaling cascades are transient by necessity.

### SUMMARY OF THE INVENTION

Accordingly, the inventor has utilized novel combinatorial libraries to identify ligands of SH3 domains and inhibitors of proteins comprising SH3 domains.

Thus, in some embodiments, the invention is directed to compounds comprising A-Arg-B-Leu-Pro-Pro-Leu-Pro-C,
where A=moiety I, 11, or III

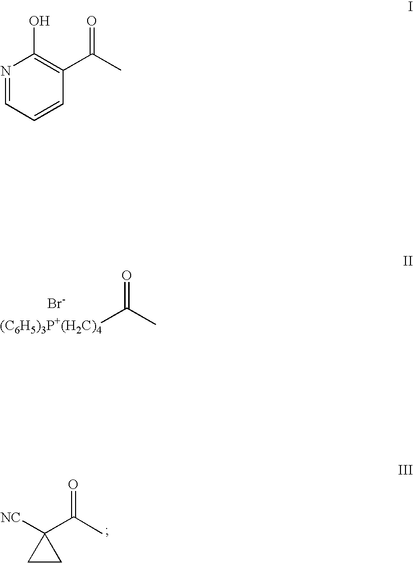

B=Ala, (L)-2,3-diaminopropionic acid (Dap) or

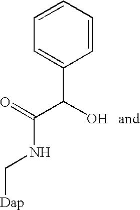

C=any moiety. In these embodiments, any amino acid can alternatively be an analogous peptidomimetic.

In other embodiments, the invention is directed to combinatorial libraries comprising a plurality of compounds, each compound comprising A-Arg-B-Leu-Pro-Pro-Leu-Pro-C. In these embodiments,

A is H, NH₂ or an organic compound less than about 500 Dalton,

B is Ala, Dap, or Dap-D, where D is an organic compound less than about 500 Dalton, and

C is any moiety. In these embodiments, any amino acid can alternatively be an analogous peptidomimetic. Each of the compounds in these embodiments is different.

The present invention is also directed to methods of identifying a compound that binds to an SH3 domain. The methods comprise

creating a first combinatorial library as described above;

screening the compounds in the first combinatorial library for binding to the SH3 domain; and

identifying any compounds in the first combinatorial library that bind to the SH3 domain.

In additional embodiments, the invention is directed to methods of inhibiting an activity of a protein comprising an SH3 domain. The methods comprise identifying a compound that inhibits the SH3 domain by the method described above, then contacting the compound with the protein in a manner sufficient to inhibit the activity of the protein.

The invention is further directed to methods of inhibiting the activity of a protein comprising an SH3 domain. The methods comprise combining the protein with a compound described above that inhibits the protein, in a manner sufficient to inhibit the activity of the protein.

In further embodiments, the invention is directed to methods of treating a mammal having a deleterious condition that is mediated by a protein comprising an SH3 domain. The methods comprise administering a pharmaceutical composition to the mammal, where the composition comprises a compound as described above that inhibits the protein.

The invention is also directed to the use of a compound that binds to the SH3 domain of a protein kinase in the manufacture of a medicament for the treatment of a deleterious condition in a mammal that is dependent on a protein kinase for induction or severity. In these embodiments, the treatment comprises contacting the mammal with a pharmaceutical composition that comprises a compound as described above that inhibits the protein.

### BRIEF DESCRIPTION OF THE DRAWINGS

FIG. 1 is an outline of the iterative library-based strategy. The protocol seeks to identify potential interaction sites that circumscribe the peptide binding on the SH3 domain that are otherwise inaccessible to conventional amino acid residues. The final synthetic step, namely cleavage of the modified peptide from the resin with assay buffer, delivers the library in an assay ready form.

FIG. 2 shows the primary (Library I), secondary (Libraries II-IV), and tertiary (Library V) libraries where a=Pd, b=1% CF₃CO₂H, c=720 RCO₂H, and d=assay buffer containing dithiothreitol.

FIG. 3 shows peptoid 6, which was based on the previously described peptoid 5 (Nguyen et al., 2000). Peptoid 6 was used for the ELISA screen described in the Example. The fluorescently labeled peptoid 7 exhibits a K_{D} of 230±30 nM for the Fyn SH3 domain.

FIG. 4 shows parent peptide 9 and primary (8), secondary (11), and tertiary (14) leads derived from Libraries I, IV, and V, respectively.

FIG. 5 shows a chemiluminescent Western blot showing the inhibition of the WASP-Fyn SH3 interaction with the Fyn-SH3 domain ligand 14. vGlutathione sepharose beads saturated with Fyn-SH3 were incubated with U937 cell lysates in the presence of inhibitory peptide 14 or acetylated control 9 at the appropriate concentrations (μM).

### DETAILED DESCRIPTION OF THE INVENTION

The inventor has developed novel combinatorial libraries for identifying ligands of SH3 domains. Using these libraries, the inventor has identified several SH3 ligands and inhibitors of proteins having SH3 ligands, some of which have specificity for particular SH3 ligands.

Thus, in some embodiments, the present invention is directed to compounds comprising A-Arg-B-Leu-Pro-Pro-Leu-Pro-C,
wherein A=moiety I, II, or III

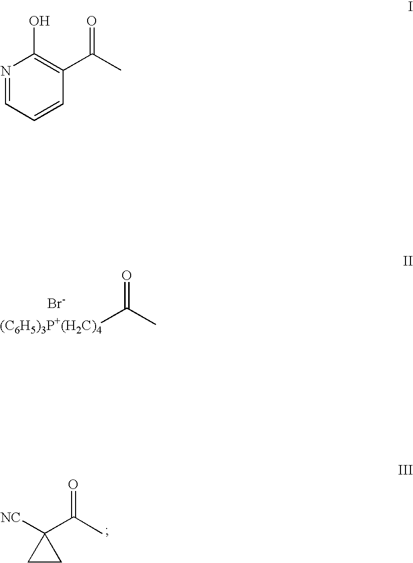

B=Ala, (L)-2,3-diaminopropionic acid (Dap) or

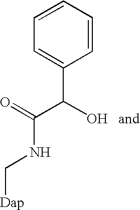

C=any moiety. In these compounds, any of amino acid can alternatively be an analogous peptidomimetic.

As used herein, a peptidomimetic is a compound that is capable of mimicking a natural parent amino acid in a protein, in that the peptidomimetic does not affect the activity of the protein. Proteins comprising peptidomimetics are generally not substrates of proteases and are likely to be active in vivo for a longer period of tine as compared to the natural proteins. In addition, they could be less antigenic and show an overall higher bioavailability. The skilled artisan would understand that design and synthesis of peptidomimetics that could substitute for any particular oligopeptide (such as the inhibitors of this invention) would not require undue experimentation. See, e.g., Ripka et al., 1998; Kieber-Emmons et al., 1997; Sanderson, 1999. Included as a peptidomimetic for several amino acids (e.g., Lys, Pro and Ala) is (L)-2,3-diaminopropionic acid (Dap).

In preferred embodiments, C is NH₂, an organic compound less than 500 Dalton or an organic compound less than 500 Dalton and a resin. A resin would normally be used as an aid to synthesize the compound as part of a combinatorial library, as in the Example below. These embodiments are not limited to the use of any particular resin. The skilled artisan could identify and utilize a useful resin for any particular purpose without undue experimentation.

In more preferred embodiments, C is NH₂,

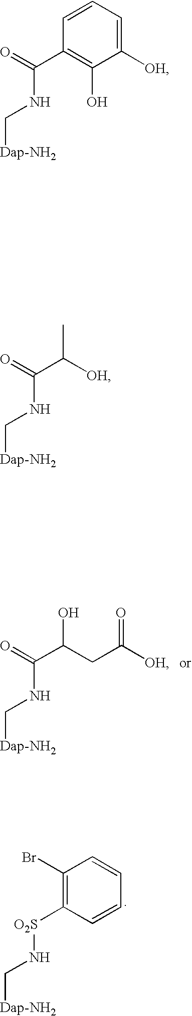

In the most preferred embodiments, C is

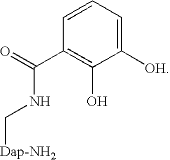

With regard to A, the most preferred moiety is I. In additional preferred embodiments, A is moiety I and C is

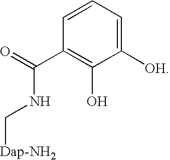

With regard to B, the most preferred embodiment is

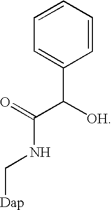

In additional preferred embodiments, the compound comprises

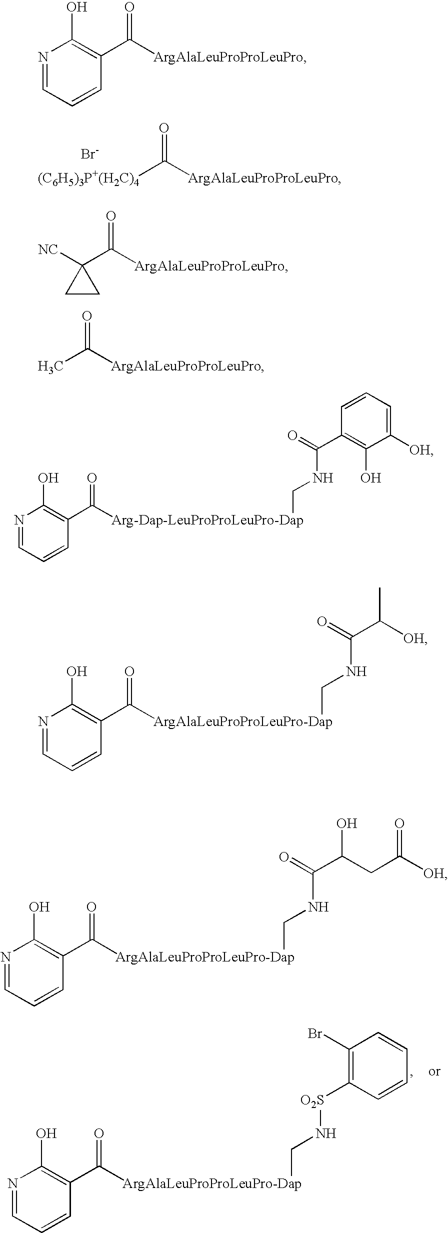

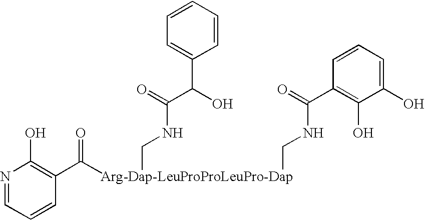

Preferably, the compound consists of

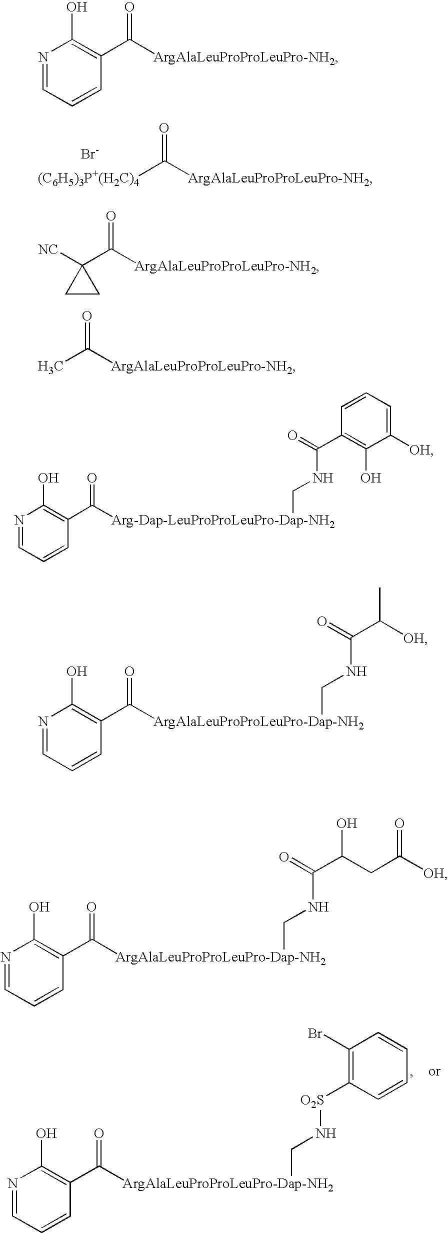

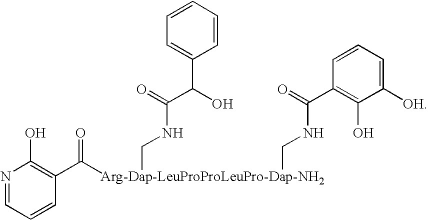

More preferably, the compound comprises

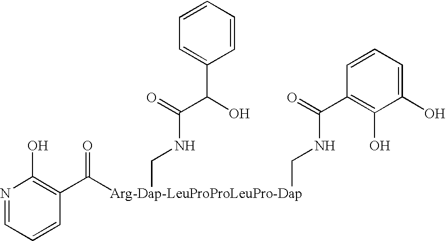

In the most preferred embodiments, the compound is

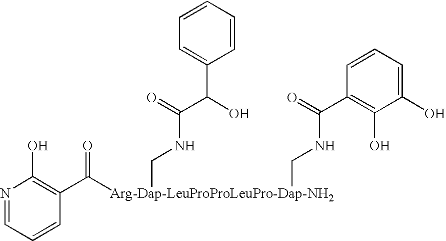

### (see Example).

Preferred compounds of these embodiments are capable of binding an SH3 domain. Preferably, these SH3 domain-binding compounds inhibit activity of a protein comprising the SH3 domain. In other preferred embodiments, the SH3 domain is naturally occurring. In more preferred embodiments, the SH3 domain is part of a vertebrate protein. Even more preferably, the SH3 domain is part of a mammalian protein; in most preferred embodiments, the mammalian protein is a human protein.

Where the compound is capable of binding an SH3 domain, the SH3 domain can be part of a signaling protein. The SH3 domain can also be is part of a protein kinase, for example an Src family member, e.g., Fyn, Src, Fgr, Yes, Yrk, Lyn, Hck, Lck, or Blk. A preferred Src family member is Fyn.

The methods described in the Example are capable of identifying ligands of SH3 domains that bind more tightly (i.e., have lower dissociation constants) than previously described SH3 ligands. Thus, the compounds of these embodiments that are SH3 domain ligands preferably have a dissociation constant (K_{D}) for the SH3 domain that is <50 μM. More preferably, the compound has a dissociation constant (K_{D}) for the SH3 domain is <2 μM. In the most preferred embodiments, the dissociation constant (K_{D}) for the SH3 domain is <0.5 μM.

The methods described in the Example are also capable of identifying ligands of SH3 domains that selectively bind to some SH3 domains but not others. Thus, the compounds of these embodiments that are SH3 domain ligands preferably have a lower dissociation constant for one group of SH3 domains than for another group of SH3 domains. In some of these embodiments, the compound has a lower dissociation constant for an SH3 domain of a Group A Src kinase family member than for another SH3 domain. Preferably, the Group A Src kinase family member is Fyn, Yes, or Src. The another SH3 domain can be any non-Group A SH3 domain, for example a Group B Src family member, e.g., Lck or Hck. In other embodiments, the binding to the SH3 domain has a lower dissociation constant for an SH3 domain of a Group B Src kinase family member, than for another SH3 domain, e.g., a Group A Src kinase family member.

The invention is also directed to compositions comprising any of the above-described compounds that bind in a pharmaceutically acceptable carrier. Compounds that bind to SH3 domains can be treatments for disease by affecting activity of a protein comprising the SH3 domains, where the protein is involved in the disease.

By “pharmaceutically acceptable” it is meant a material that (i) is compatible with the other ingredients of the composition without rendering the composition unsuitable for its intended purpose, and (ii) is suitable for use with subjects as provided herein without undue adverse side effects (such as toxicity, irritation, and allergic response). Side effects are “undue” when their risk outweighs the benefit provided by the composition. Non-limiting examples of pharmaceutically acceptable carriers include, without limitation, any of the standard pharmaceutical carriers such as phosphate buffered saline solutions, water, emulsions such as oil/water emulsions, microemulsions, and the like.

The above-described compounds can be formulated without undue experimentation for administration to a mammal, including humans, as appropriate for the particular application. Additionally, proper dosages of the compositions can be determined without undue experimentation using standard dose-response protocols.

Accordingly, the compositions designed for oral, lingual, sublingual, buccal and intrabuccal administration can be made without undue experimentation by means well known in the art, for example with an inert diluent or with an edible carrier. The compositions may be enclosed in gelatin capsules or compressed into tablets. For the purpose of oral therapeutic administration, the pharmaceutical compositions of the present invention may be incorporated with excipients and used in the form of tablets, troches, capsules, elixirs, suspensions, syrups, wafers, chewing gums and the like.

Tablets, pills, capsules, troches and the like may also contain binders, recipients, disintegrating agent, lubricants, sweetening agents, and flavoring agents. Some examples of binders include microcrystalline cellulose, gum tragacanth or gelatin. Examples of excipients include starch or lactose. Some examples of disintegrating agents include alginic acid, cornstarch and the like. Examples of lubricants include magnesium stearate or potassium stearate. An example of a glidant is colloidal silicon dioxide. Some examples of sweetening agents include sucrose, saccharin and the like. Examples of flavoring agents include peppermint, methyl salicylate, orange flavoring and the like. Materials used in preparing these various compositions should be pharmaceutically pure and nontoxic in the amounts used.

The compounds can easily be administered parenterally such as for example, by intravenous, intramuscular, intrathecal or subcutaneous injection. Parenteral administration can be accomplished by incorporating the compounds into a solution or suspension. Such solutions or suspensions may also include sterile diluents such as water for injection, saline solution, fixed oils, polyethylene glycols, glycerine, propylene glycol or other synthetic solvents. Parenteral formulations may also include antibacterial agents such as for example, benzyl alcohol or methyl parabens, antioxidants such as for example, ascorbic acid or sodium bisulfite and chelating agents such as EDTA. Buffers such as acetates, citrates or phosphates and agents for the adjustment of tonicity such as sodium chloride or dextrose may also be added. The parenteral preparation can be enclosed in ampules, disposable syringes or multiple dose vials made of glass or plastic.

Rectal administration includes administering the compound, in a pharmaceutical composition, into the rectum or large intestine. This can be accomplished using suppositories or enemas. Suppository formulations can easily be made by methods known in the art. For example, suppository formulations can be prepared by heating glycerin to about 120° C., dissolving the composition in the glycerin, mixing the heated glycerin after which purified water may be added, and pouring the hot mixture into a suppository mold.

Transdermal administration includes percutaneous absorption of the composition through the skin. Transdermal formulations include patches (such as the well-known nicotine patch), ointments, creams, gels, salves and the like.

The present invention includes nasally administering to the mammal a therapeutically effective amount of the compound. As used herein, nasally administering or nasal administration includes administering the compound to the mucous membranes of the nasal passage or nasal cavity of the patient. As used herein, pharmaceutical compositions for nasal administration of the compound include therapeutically effective amounts of the compound prepared by well-known methods to be administered, for example, as a nasal spray, nasal drop, suspension, gel, ointnent, cream or powder. Administration of the compound may also take place using a nasal tampon or nasal sponge.

Where the compound is administered peripherally such that it must cross the blood-brain barrier to access the target protein, the compound is preferably formulated in a pharmaceutical composition that enhances the ability of the compound to cross the blood-brain barrier of the mammal. Such formulations are known in the art and include lipophilic compounds to promote absorption. Uptake of non-lipophilic compounds can be enhanced by combination with a lipophilic substance. Lipophilic substances that can enhance delivery of the compound across the nasal mucus include but are not limited to fatty acids (e.g., palmitic acid), gangliosides (e.g., GM-I), phospholipids (e.g., phosphatidylserine), and emulsifiers (e.g., polysorbate 80), bile salts such as sodium deoxycholate, and detergent-like substances including, for example, polysorbate 80 such as Tween™, octoxynol such as Triton™ X-100, and sodium tauro-24,25-dihydrofusidate (STDHF). See Lee et al., Biopharm., April 1988 issue:3037.

In particular embodiments of the invention, the compound is combined with micelles comprised of lipophilic substances. Such micelles can modify the permeability of the nasal membrane to enhance absorption of the compound. Suitable lipophilic micelles include without limitation gangliosides (e.g., GM-I ganglioside), and phospholipids (e.g., phosphatidylserine). Bile salts and their derivatives and detergent-like substances can also be included in the micelle formulation. The compound can be combined with one or several types of micelles, and can further be contained within the micelles or associated with their surface.

Alternatively, the compound can be combined with liposomes (lipid vesicles) to enhance absorption. The compound can be contained or dissolved within the liposome and/or associated with its surface. Suitable liposomes include phospholipids (e.g., phosphatidylserine) and/or gangliosides (e.g., GM-1). For methods to make phospholipid vesicles, see for example, U.S. Pat. No. 4,921,706 to Roberts et al., and U.S. Pat. No. 4,895,452 to Yiournas et al. Bile salts and their derivatives and detergent-like substances can also be included in the liposome formulation.

In preferred embodiments, the compound in these pharmaceutical compositions inhibits activity of a protein comprising the SH3 domain. Preferably, the SH3 domain is part of a mammalian protein, most preferably a human protein. In some embodiments, the SH3 domain is part of a signaling protein. The SH3 domain can also be part of a protein kinase, e.g., an Src family member such as Fyn, Src, Fgr, Yes, Yrk, Lyn, Hck; Lck, or Blk. Preferably, the Src family member is Fyn.

In other preferred embodiments, the compound in these pharmaceutical compositions comprise

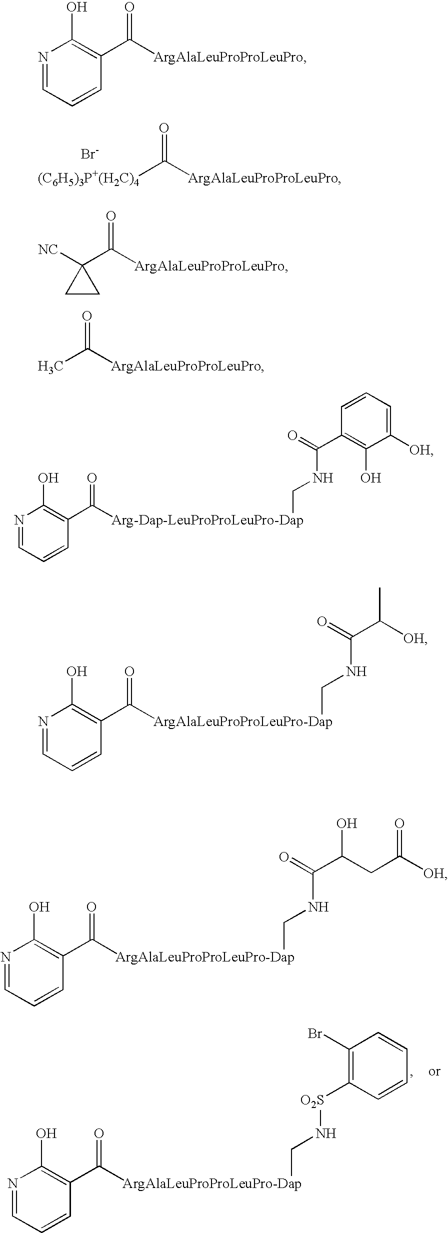

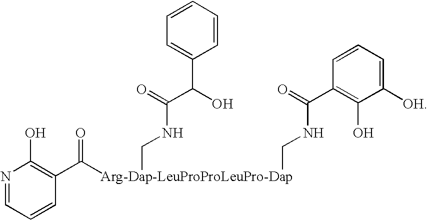

Preferably, the compound consists of

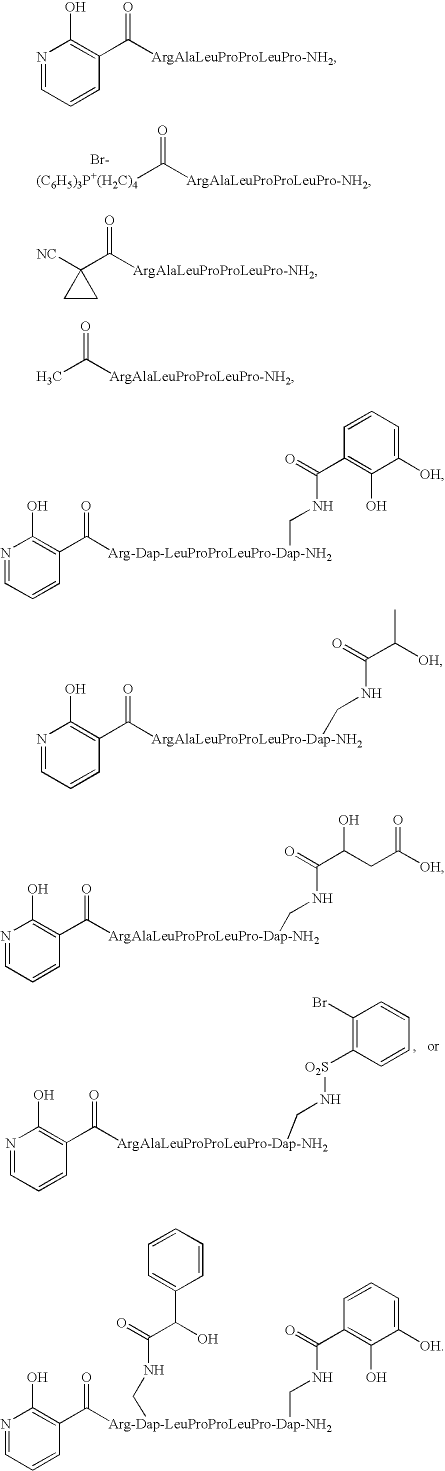

In more preferred embodiments, the compound in the pharmaceutical composition comprises

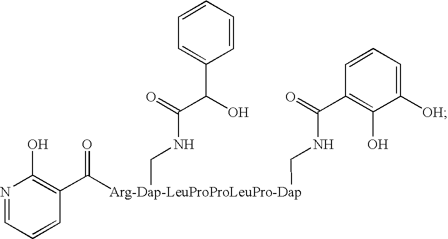

most preferably the compound consists of

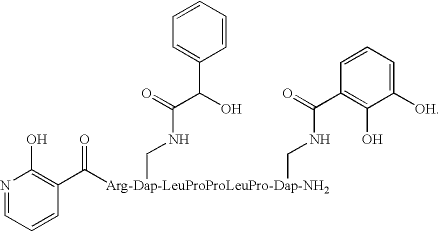

In other embodiments, the pharmaceutical composition is formulated in unit dosage for treatment of a deleterious condition in a mammal. Preferably, the mammal is a human. In other preferred embodiments, the deleterious condition is mediated by an Src family member. Nonlimiting examples of the deleterious condition is a cancer, a cardiovascular disease, an inflammatory disease, an autoimmune disease, a destructive bone disorder, a proliferative disorder, a neurological disease, a neurodegenerative disease, reperfusion/ischemia in stroke, an anigiogenic disorder, an allergy, an arthritis, or an infectious disease.

In some embodiments, the deleterious condition is a Fyn- or Src-mediated disease or condition, e.g., hypercalcemia, restenosis, osteoporosis, osteoarthritis, symptomatic treatment of bone metastasis, rheumatoid arthritis, inflammatory bowel disease, multiple sclerosis, psoriasis, lupus, graft vs. host disease, T-cell mediated hypersensitivity disease, Hashimoto's thyroiditis, Guillain-Barre syndrome, chronic obstructive pulmonary disorder, contact dermatitis, cancer, Paget's disease, asthma, ischemic or reperfusion injury, allergic disease, atopic dermatitis, allergic rhinitis, autoimmune diseases, or leukemia.

Where the deleterious condition is cancer, non-limiting examples include carcinoma (such as squamous cell carcinoma, small cell lung cancer, cancer of the bladder, breast, colon, kidney, liver, lung, esophagus, gall-bladder, ovaly, pancreas, stomach, cervix, thyroid, prostate, or skin), hematopoietic tumor of lymphoid lineage (such as leukemias, acute lymphocytic. leukemia, acute lymphoblastic leukemia, B-cell lymphoma, T-cell lymphoma, Hodgkin's lymphoma, non-Hodgkin's lymphoma, hairy cell lymphoma or Burkett's lymphoma), hematopoietic tumor of myeloid lineage (such as acute and chronic myelogenous leukemias, myelodysplastic syndrome or promyelocytic leukemia), tumor of mesenchymal origin (such as fibrosarcomas, rhabdomyosarcoma, soft tissue sarcomas or bone sarcomas), tumor of the central and peripheral nervous system (such as astrocytomas, neuroblastomas, gliomas or schwannomas), and other tumor (such as melanomas, seminomas, teratocarcinomas, osteosarcomas, xeroderma pigmentosum, keratoacanthoma, thyroid follicular cancer or Kaposi's sarcoma).

Where the deleterious condition is an autoimmune disease, non-limiting examples include glomerulonephritis, rheumatoid arthritis, systemic lupus erythematosus, scleroderma, chronic thyroiditis, Graves' disease, autoimmune gastritis, diabetes, autoimmune hemolytic anemia, autoimmune neutropenia, thrombocytopenia, atopic dennatitis, chronic active hepatitis, myasthenia gravis, multiple sclerosis, inflammatory bowel disease, ulcerative colitis, Crohn's disease, psoriasis, and graft vs. host disease.

Where the deleterious condition is a destructive bone disorder, non-limiting examples include osteoarthritis, osteoporosis and multiple myeloma-related bone disorder. Where the deleterious condition is a proliferative disorder, non-limiting examples include acute myelogenous leukemia, chronic myelogenous leukemia, metastatic melanoma, Kaposi's sarcoma, and multiple myeloma. Where the deleterious condition is a neurological or neurodegenerative disease, non-limiting examples include Alzheimer's disease, Parkinson's disease, amyotrophic lateral sclerosis, Huntington's disease, cerebral ischemia or neurodegenerative disease caused by traumatic injury, glutamate neurotoxicity and hypoxia. Where the deleterious condition is ischemia/reperfusion injury, non-limiting examples include ischemia/reperfusion in stroke, myocardial ischemia, renal ischemia, heart attack, organ hypoxia or thrombin-induced platelet aggregation.

In preferred embodiments, the deleterious condition is hypercalcemia, restenosis, osteoporosis, osteoarthritis, symptomatic treatment of bone metastasis, rheumatoid arthritis, inflammatory bowel disease, multiple sclerosis, psoriasis, lupus, graft vs. host disease, T-cell mediated hypersensitivity disease, Hashimoto's thyroiditis, Guillain-Barre syndrome, chronic obstructive pulmonary disorder, contact dermatitis, Paget's disease, asthma, ischemic or reperfusion injury, allergic disease, atopic dermatitis, allergic rhinitis, a carcinoma a hematopoietic tumor of lymphoid lineage, a hematopoietic tumor of myeloid lineage, a tumor of mesenchymal origin, a tumor of the nervous system, a melanoma, a seminoma, a teratocarcinoma, an osteosarcoma, xeroderma pigmentosum, keratoacanthoma, thyroid follicular cancer, Kaposi's sarcoma, acute pancreatitis, chronic pancreatitis, asthma, allergies, adult respiratory distress syndrome, glomerulonephritis, rheumatoid arthritis, systemic lupus erythematosus, scleroderma, chronic thyroiditis, Graves' disease, autoimmune gastritis, diabetes, autoimmune hemolytic anemia, autoimmune neutropenia, thrombocytopenia, atopic dermatitis, chronic active hepatitis, myasthenia gravis, multiple sclerosis, inflammatory bowel disease, ulcerative colitis, Crohn's disease, psoriasis, osteoarthritis, osteoporosis, acute myelogenous leukemia, chronic myelogenous leukemia, metastatic melanoma, Kaposi's sarcoma, multiple myeloma, Alzheimer's disease, Parkinson's disease, amyotrophic lateral sclerosis, Huntington's disease, cerebral ischemia, neurodegenerative disease caused by traumatic injury, glutamate neurotoxicity, ischemia/reperfusion injury, renal ischemia, heart attack, organ hypoxia, or thrombin-induced platelet aggregation.

The present invention is also directed to combinatorial libraries comprising a plurality of compounds, each compound comprising A-Arg-B-Leu-Pro-Pro-Leu-Pro-C, wherein

A is H, NH₂ or an organic compound less than about 500 Dalton,

B is Ala, Dap, or Dap-D, where D is an organic compound less than about 500 Dalton, and

C is any moiety, and wherein any amino acid can alternatively be an analogous peptidomimetic. In these libraries, each compound is different.

In preferred embodiments, C is H, NH₂, Pro, Dap or Dap-E, where E is an organic compound less than about 500 Dalton.

In other preferred embodiments, at least one of A, B or C varies among compounds by comprising different acyl groups.

In additional preferred embodiments, each compound comprises RHN-ArgAlaLeuProProLeuPro, where each compound has a different R, preferably a different acyl group.

In more preferred embodiments, each compound comprises

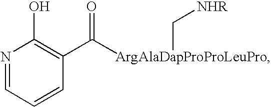

where each compound has a different R; preferably each R is a different acyl group. In other more preferred embodiments, each compound comprises

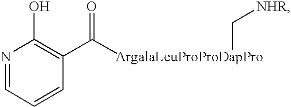

where each compound has a different R, preferably a different acyl group. In additional more preferred embodiments, each compound comprises

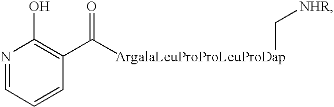

where each compound has a different R, preferably a different acyl group.

In even more preferred embodiments, each compound comprises

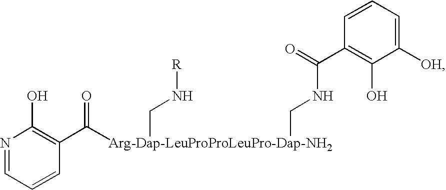

where each compound has a different R, preferably a different acyl group

The combinatorial library of these embodiments can comprise any number of compounds, e.g., 2, 10, 100, 500, or more.

The present invention is additionally directed to methods of identifying a compound that binds to an SH3 domain, the methods comprising

creating a first combinatorial library as described above;

screening the compounds in the first combinatorial library for binding to the SH3 domain; and

identifying any compounds in the first combinatorial library that bind to the SH3 domain. See Example for applications of these methods.

If desired, any compounds identified by these methods that bind to the SH3 domain can be screened for preferential selectivity for the SH3 domain. Additionally or alternatively, identified compounds can be screened for the ability to inhibit an activity of a protein comprising the SH3 domain.

These methods are preferably iterative, i.e., they can further comprise

selecting a first compound that binds to the SH3 domain from the first combinatorial library, and

creating a second combinatorial library as described above,
wherein the second combinatorial library comprises
   the A, B, or C from the first compound that varied in the first combinatorial library, and
   another A, B, or C that varies among the compounds in the second combinatorial library. The members of this second round of combinatorial libraries are then preferably screened for binding to the SH3 domain that is stronger than the first compound that served as the source of the second combinatorial library round. Additionally or alternatively, the members of the second round of combinatorial libraries can be screened for preferential selectivity for the SH3 domain.

Preferred combinatorial libraries for these methods include RHN-ArgAlaLeuProProLeuPro,

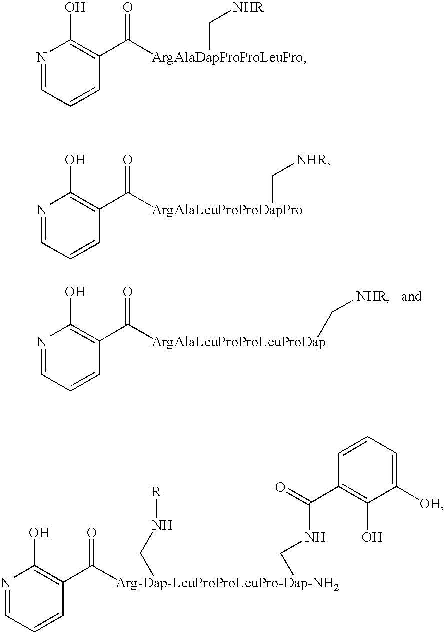

where each compound has a different R.

In additional embodiments, the invention is directed to methods of inhibiting an activity of a protein comprising an SH3 domain. The methods comprise identifying a compound that inhibits the SH3 domain by the above-described methods using a combinatorial library, then contacting the compound with the protein in a manner sufficient to inhibit the activity of the protein.

In these embodiments, the protein is preferably a protein kinase, more preferably an Src family member, such as Fyn, Src, Fgr, Yes, Yrk, Lyn, Hck, Lck, or Blk, most preferably Fyn. In other preferred embodiments, the protein is in a mammalian cell, e.g., a human cell. The mammalian cell can also be part of a live mammal, preferably a human. The live mammal can also be at risk for a deleterious condition mediated by the protein. Alternatively, the live mammal can have a deleterious condition mediated by the protein. In these embodiments, the deleterious condition is preferably mediated by an Src family member. Examples of such deleterious conditions are cancer, a cardiovascular disease, an inflammatory disease, an autoimmune disease, a destructive bone disorder, a proliferative disorder, a neurological disease, a neurodegenerative disease, reperfusion/ischemia in stroke, an angiogenic disorder, an allergy, an arthritis, and an infectious disease. Preferably, the deleterious condition is hypercalcemia, restenosis, osteoporosis, osteoarthritis, symptomatic treatment of bone metastasis, rheumatoid arthritis, inflammatory bowel disease, multiple sclerosis, psoriasis, lupus, graft vs. host disease, T-cell mediated hypersensitivity disease, Hashimoto's thyroiditis, Guillain-Barre syndrome, chronic obstructive pulmonary disorder, contact dermatitis, Paget's disease, asthma, ischemic or reperfusion injury, allergic disease, atopic dermatitis, allergic rhinitis, a carcinoma, a hematopoietic tumor of lymphoid lineage, a hematopoietic tumor of myeloid lineage, a tumor of mesenchymal origin, a tumor of the nervous system, a melanoma, a seminoma, a teratocarcinoma, an osteosarcoma, xeroderma pigmentosum, keratoacanthoma, thyroid follicular cancer, Kaposi's sarcoma, acute pancreatitis, chronic pancreatitis, asthma, allergies, adult respiratory distress syndrome, glomerulonephritis, rheumatoid arthritis, systemic lupus erythematosus, sclerodermia, chronic thyroiditis, Graves' disease, autoimmune gastritis, diabetes, autoimmune hemolytic anemia, autoimmune neutropenia, thrombocytopenia, atopic dermatitis, chronic active hepatitis, myasthenia gravis, multiple sclerosis, inflammatory bowel disease, ulcerative colitis, Crohn's disease, psoriasis, osteoarthritis, osteoporosis, acute myelogenous leukemia, chronic myelogenous leukemia, metastatic melanoma, Kaposi's sarcoma, multiple myeloma, Alzheimer's disease, Parkinson's disease, amyotrophic lateral sclerosis, Huntington's disease, cerebral ischemia, neurodegenerative disease caused by traumatic injury, glutamate neurotoxicity, ischemia/reperfusion injury, renal ischemia, heart attack, organ hypoxia, or thrombin-induced platelet aggregation.

The invention is also directed to methods of inhibiting the activity of a protein comprising an SH3 domain. The methods comprise combining the protein with a compound that inhibits the activity of the protein as described above in a manner sufficient to inhibit the activity of the protein. Preferably, the SH3 domain is naturally occurring. In other preferred embodiments, the SH3 domain is part of a mammalian protein, for example a human protein.

The protein of these embodiments is preferably a protein kinase, more preferably an Src family member, such as Fyn, Src, Fgr, Yes, Yrk, Lyn, Hck, Lck, or BIk, most preferably Fyn. In other preferred embodiments, the protein is in a mammalian cell, e.g., a human cell. The mammalian cell can also be part of a live mammal, preferably a human. The live mammal can also be at risk for a deleterious condition mediated by the protein. Alternatively, the live mammal can have a deleterious condition mediated by the protein. In these embodiments, the deleterious condition is preferably mediated by an Src family member. Examples of such deleterious conditions are cancer, a cardiovascular disease, an inflammatory disease, an autoimmune disease, a destructive bone disorder, a proliferative disorder, a neurological disease, a neurodegenerative disease, reperfusion/ischemia in stroke, an angiogenic disorder, an allergy, an arthritis, and an infectious disease. Preferably, the deleterious condition is hypercalcemia, restenosis, osteoporosis, osteoarthritis, symptomatic treatment of bone metastasis, rheumatoid arthritis, inflammatory bowel disease, multiple sclerosis, psoriasis, lupus, graft vs. host disease, T-cell mediated hypersensitivity disease, Hashimoto's thyroiditis, Guillain-Barre syndrome, chronic obstructive pulmonary disorder, contact dermatitis, Paget's disease, asthma, ischemic or reperfusion injury, allergic disease, atopic dermatitis, allergic rhinitis, a carcinoma, a hematopoietic tumor of lymphoid lineage, a hematopoietic tumor of myeloid lineage, a tumor of mesenchymal origin, a tumor of the nervous system, a melanoma, a seminoma, a teratocarcinoma, an osteosarcoma, xeroderma pigmentosum, keratoacanthoma, thyroid follicular cancer, Kaposi's sarcoma, acute pancreatitis, chronic pancreatitis, asthma, allergies, adult respiratory distress syndrome, glomerulonephritis, rheumatoid arthritis, systemic lupus erythematosus, scleroderma, chronic thyroiditis, Graves' disease, autoimmune gastritis, diabetes, autoimmune hemolytic anemia, autoimmune neutropenia, thrombocytopenia, atopic dennatitis, chronic active hepatitis, myasthenia gravis, multiple sclerosis, inflammatory bowel disease, ulcerative colitis Crohn's disease, psoriasis, osteoarthritis, osteoporosis, acute myelogenous leukemia, chronic myelogenous leukemia, metastatic melanoma, Kaposi's sarcoma, multiple myeloma, Alzheimer's disease, Parkinson's disease, amyotrophic lateral sclerosis, Huntington's disease, cerebral ischemia, neurodegenerative disease caused by traumatic injury, glutamate neurotoxicity, ischemia/reperfusion injury, renal ischemia, heart attack, organ hypoxia, or thrombin-induced platelet aggregation.

The compound of these embodiments preferably comprises

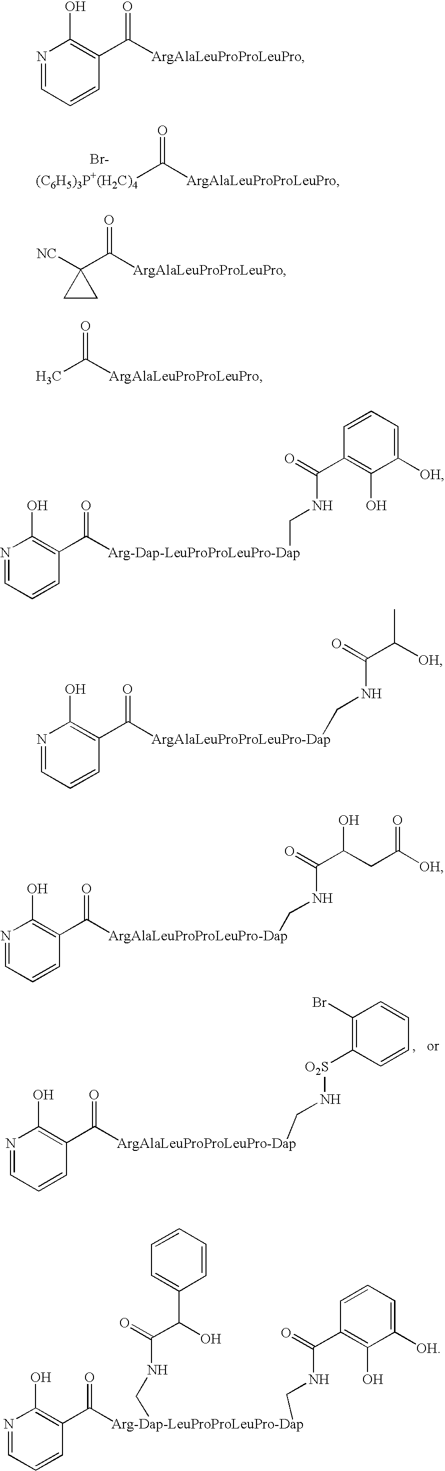

More preferably, the compound consists of

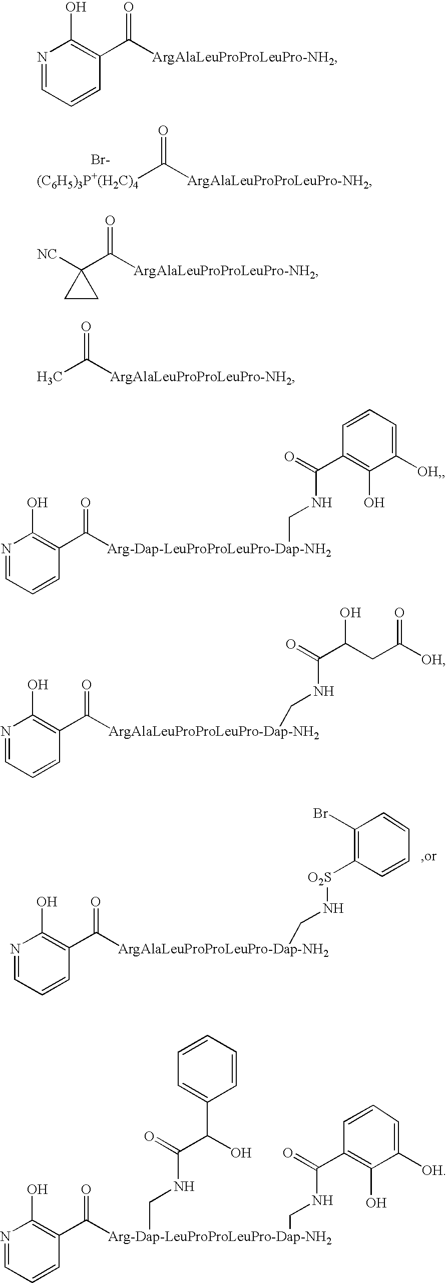

Even more preferably, the compound comprises

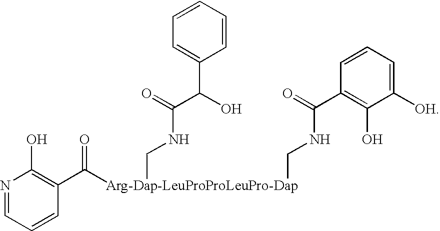

In the most preferred embodiments, the compound consists of

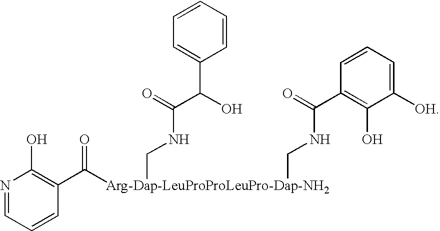

The instant invention is additionally directed to methods of treating a mammal having a deleterious condition that is mediated by a protein comprising an SH3 domain. The methods comprise administering a compound in a pharmaceutical carrier to the mammal, where the compound is one of the above-described compounds that bind to the protein. Preferably, the mammal of these embodiments is a human.

The protein is of these embodiments is preferably a protein kinase, more preferably an Src family member, such as Fyn, Src, Fgr, Yes, Yrk, Lyn, Hck, Lck, or Blk, most preferably Fyn. In more preferred embodiments, the compound comprises

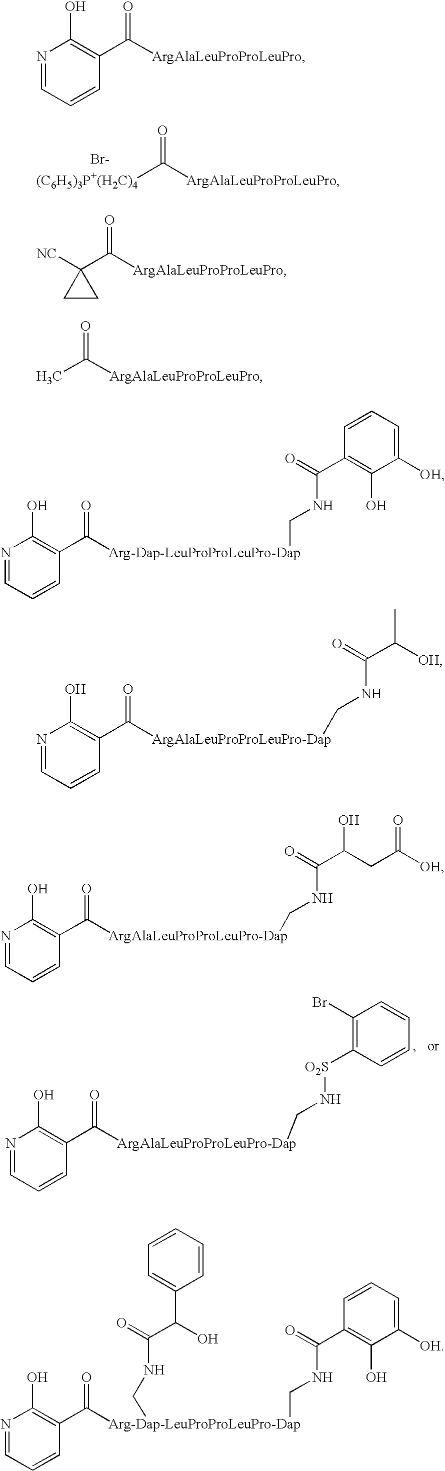

More preferably, the compound consists of

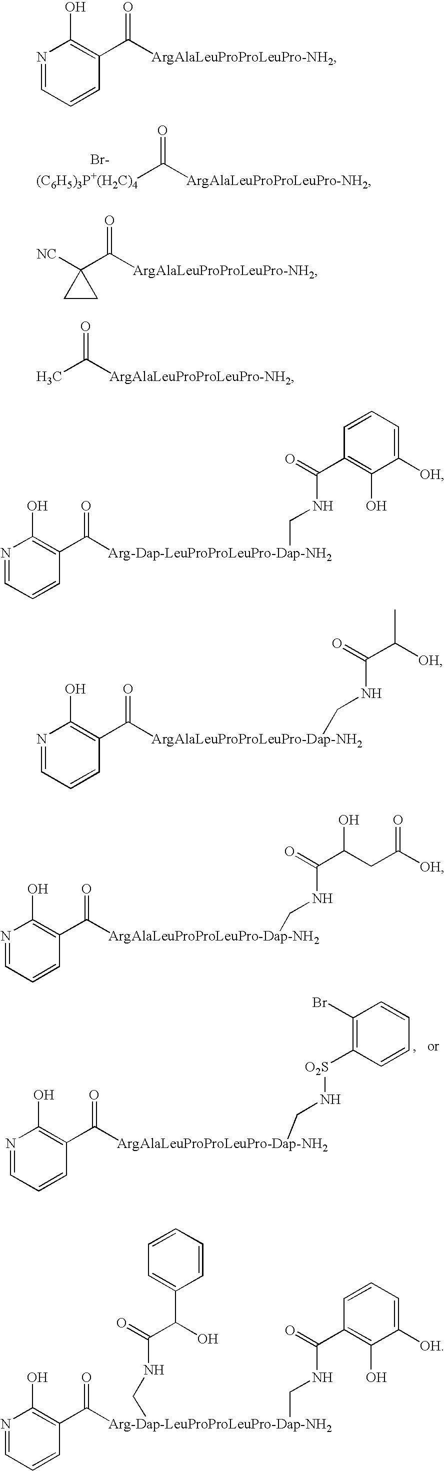

In additional embodiments, the invention is directed to the use of a compound that binds to the SH3 domain of a protein kinase in the manufacture of a medicament for the treatment of a deleterious condition in a mammal that is dependent on a protein kinase for induction or severity. The treatment in these embodiments comprises contacting the mammal with a pharmaceutical composition that comprises a compound described above that binds to the SH3 domain.

Preferably, the compound in these embodiments comprises

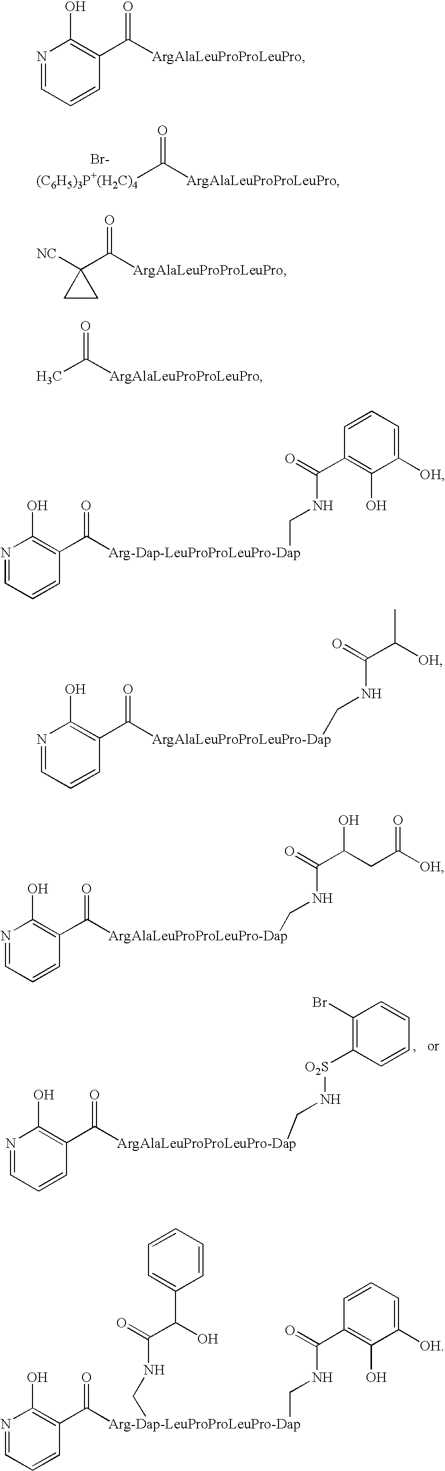

In more preferred embodiments, the compound consists of

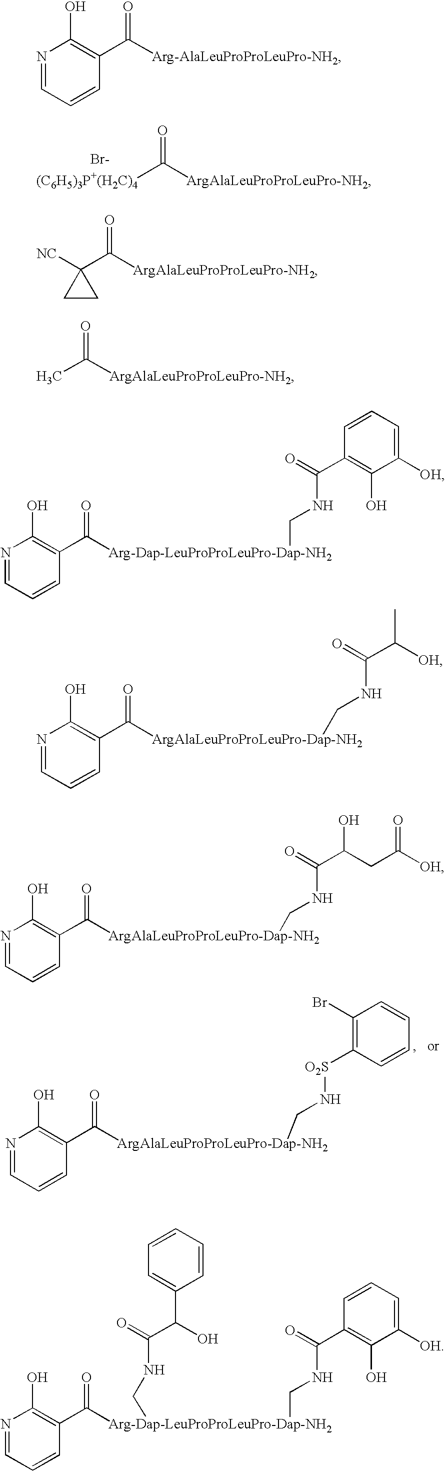

Preferred embodiments of the invention are described in the following Example. Other embodiments within the scope of the claims herein will be apparent to one skilled in the art from consideration of the specification or practice of the invention as disclosed herein. It is intended that the specification, together with the Example, be considered exemplary only, with the scope and spirit of the invention being indicated by the claims, which follow the Example.

### Example 1

### Acquisition of Fyn-Selective SH3 Domain Ligands via A Combinatorial Library Strategy

### Example Summary

A stepwise library-based strategy has been employed to acquire a potent ligand for the SH3 domain of Fyn, a Src kinase family member that plays a key role in T cell activation. The easily automated methodology is designed to identify potential interaction sites that circumscribe the protein/peptide binding region on the SH3 domain. The library protocol creates peptide/non-peptide chimeras that are able to bind to these interaction sites that are otherwise inaccessible to natural amino acid residues. The peptide-derived lead and the Fyn SH3 domain form a complex that exhibits a KD of 25±5 nM, approximately 1000-fold potent that that displayed by the corresponding conventional peptide ligand. Furthermore, the lead ligand exhibits selectivity against SH3 domains derived from other Src kinases, in spite of a sequence identity of approximately 80%.

### Introduction

We report in this study the synthesis and identification of a peptide-derived ligand that selectively targets the Fyn SH3 domain. Fyn, a member of the Src tyrosine protein kinase family, is known to play an important role in the biochemical cascade responsible for T cell activation (Palacios and Weiss, 2004). Both Fyn and its closely related counterpart, Lck, exhibit similar substrate specificities, and the SH2/SH3 domains of both proteins play key roles in thymic development and T cell proliferation in response to antigenic challenges. In particular, the SH3 domains of both have been shown to interact with the Wiskott Aldrich Syndrome protein (WASp) in vitro (Banin, 1996). To the best of our knowledge, this is the first report of a ligand that is able to discriminate between SH3 domains of the members of the Src kinase family.

### Results and Discussion

The consensus sequences for a number of SH3 domains have been reported. However, peptides (decapeptides and longer) bearing these sequences serve as relatively modest SH3 domain ligands (μM range). A number of attempts have been made to enhance the affinities of SH3 domain-directed peptides, either by searching for optimized sequences via library methods or by the introduction of non-natural substituents at the N- or C-terminii (Posern et al., 1998; Knudsen et al., 1995; Feng et al., 1996; Pisabarro, 1996). However, these strategies typically do not furnish ligands that exhibit affinities significantly beyond the μM/nM border. By contrast, Lim and his colleagues have demonstrated that N-substituted peptides (“peptoids”) exhibit high affinities and good selectivities for specific classes of SH3 domains (Nguyen et al., 1998, 2000). The critical design element in these studies is the notion that N-substituted glycine residues serve as proline mimetics and, given the diversity inherent within the former, can be screened to identify substituents that dramatically enhance SH3 domain affinity. Peptoid ligands that exhibit nM affinities were identified for the SH3 domains of Grb2 (N-terminus; K_{D}=30 nM), Src (K_{D}=140 nM), and Crk (K_{D}=8 nM)(Nguyen et al., 1998, 2000). Furthermore, these ligands exhibit a high selectivity in favor of their targeted SH3 domain in spite of the fact that these domains are structurally homologous (e.g. Crk and Src SH3 domains display a 33% sequence similarity) (Borreguero et al. 2002). These results dispel the previously held notion that it is not possible to obtain tight binding ligands for SH3 domains due to the small size (60 amino acids).

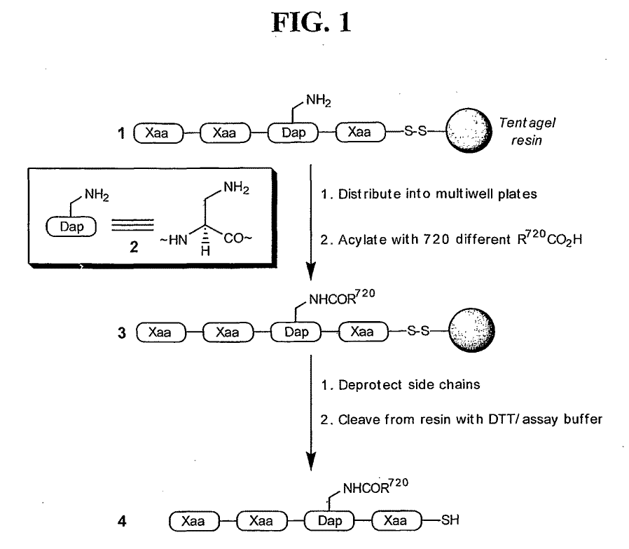

We have previously described an iterative library-based strategy that converts low affinity consensus sequence peptides into high affinity species that display pronounced selectivities for their intended protein targets (Lee and Lawrence, 1999; Yeh et al., 2001, Lee et al., 2004; Shen et al., 2001). The methodology, as illustrated in general form in FIG. 1, has been successfully applied to SH2 domains, protein tyrosine phosphatases, and protein kinases, including specific isoforms of the highly conserved protein kinase C subfamily. However, the strategy relies upon identifying a series of unique and widely interspersed interaction sites on the protein surface. Given the small size of SH3 domains, it was far from clear whether proteins of this size would be amenable to such an approach. We decided to explore this question by employing the SH3 domain of the Fyn tyrosine kinase as our target. Fyn is a member of the Src kinase family, a group of highly homologous tyrosine kinases. The extremely high sequence homology displayed by the SH3 domains from these proteins represents a serious challenge in terms of acquiring selective inhibitors (e.g. the SH3 domains of Src and Fyn are 78% identical [Plaxco et al., 1998]). To the best of our knowledge, there are no examples of ligands that distinguish between the SH3 domains of the Src kinase family.

A good portion of the binding energy that drives SH3 domain/consensus sequence complex formation results from the interaction of key residues on the consensus sequence peptide with hydrophobic pockets of the SH3 domain. However, adjacent residues can also contribute binding energy by engaging in noncovalent interactions with subsites located proximal to these lipophilic pockets. The methodology described in FIG. 1 allows one to screen for substituents, positioned on the peptide ligand, that can engage in interactions with the SH3 domain that are simply not accessible to naturally occurring amino acid residues. The consensus sequence peptide 1 was prepared on a disulfide-linked Tentagel resin. One of the residues in 1 contains (L)-2,3-diaminopropionic acid 2 (Dap), which possesses an amine handle on the side chain that can be readily modified with an array of activated carboxylic acid moieties. Peptide-resin resin 1 was then distributed, in equal amounts, into individual wells of eight 96-well synthesis plates. Each well was subsequently charged with a single carboxylic acid from a total library of 720 different commercially available carboxylic acids that vary in size, shape, polarity, and charge. Following acylation of the amine moiety on Dap (3), the side chains of the peptide were deprotected and the peptide subsequently cleaved from the resin (4) using assay buffer that contains dithiothreitol (DTT). The peptides were then filtered from the synthesis plate into a receiving plate and the 720-membered library was subsequently screened.

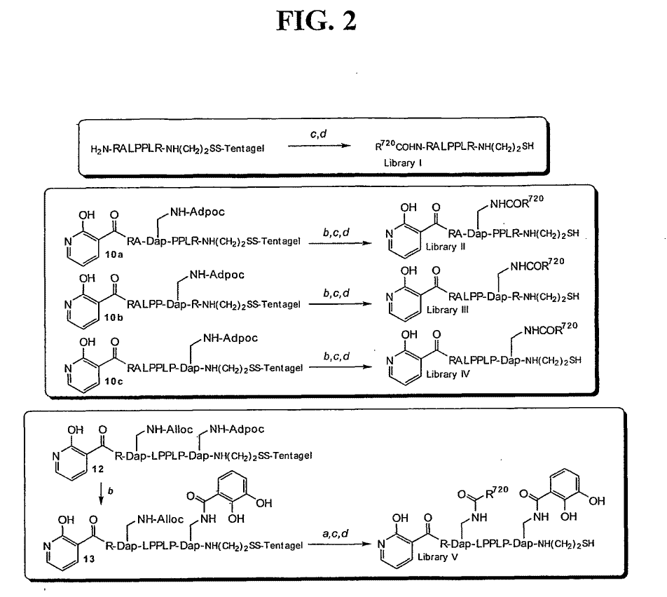

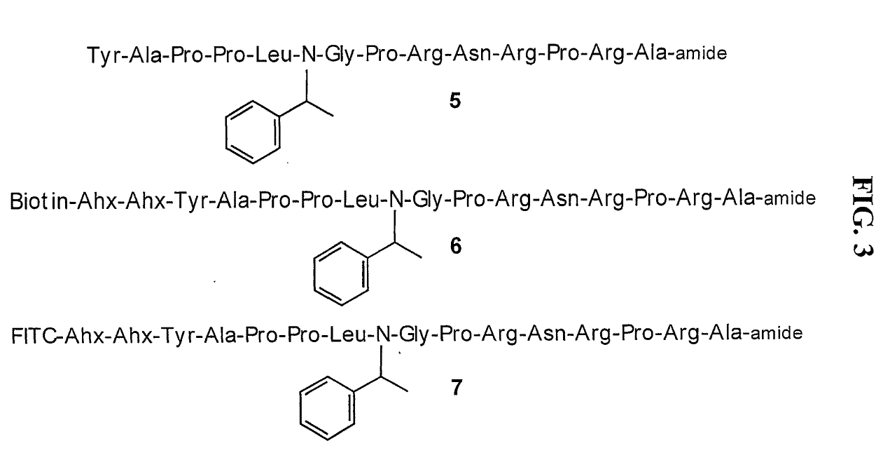

We employed Arg-Ala-Leu-Pro-Pro-Leu-Pro as the starting point for Fyn SH3 ligand optimization, a sequence that is consistent with most known SH3 domain-binding elements. We initially introduced diversity elements on the N-terminus of this peptide to furnish Library I (FIG. 2). The 720 individual members of this library were subsequently assessed for SH3 binding potency via an ELISA-based screen. The latter was performed in parallel using streptavidin-coated 96 well plates. A biotinylated-peptide SH3-directed ligand was appended to the individual wells and a GST-SH3 domain fusion protein was subsequently introduced. Competition between members of the library and the microwell-bound biotinylated peptide ligand determined the extent to which the GST-SH3 protein was retained by the individual wells after washing. The latter was quantified using an anti-GST-peroxidase fusion construct. The assay is dependent upon a strong binding interaction between the GST-SH3 domain and the biotinylated peptide ligand associated with the streptavidin-coated well. Given the relatively modest binding constants obtained with conventional peptide ligands, we decided to employ a variant of the peptoid series reported by Lim and his colleagues (Nguyen et al., 1998, 2000). Although the 13 residue-containing peptoid 5 does not discriminate between the SH3 domains of Src, Crk, and Grb2, it does exhibit a moderately good affinity for these domains (K_{D}˜200 nM). We prepared the construct 6, which contains an aminohexanoic acid (Ahx) dyad between the biotin substituent and the N-benzylated consensus sequence (FIG. 3). The Ahx₂ linker serves as a linker to ensure that the SH3 domain has spatial access to the microwell-bound peptoid moiety. We also prepared the fluorescein-labeled analogue 7, which exhibits a K_{D} of 230±30 nM for the Fyn SH3 domain, but with little selectivity versus other members of the Src kinase family (vide infra and Table 1).

TABLE 1
Dissociation constants (μM) of ligands for various Src family SH3 domains.

| Ligand | Fyn | Lck | Src | Yes | Hck |
|---|---|---|---|---|---|
| 9 | 15 ± 4 | 19 ± 8 | 21 ± 4 | 17 ± 6 | 31 ± 8 |
| 8a | 1.2 ± 0.2 | 5.3 ± 0.4 | 2.5 ± 0.2 | 2.7 ± 0.3 | 5.1 ± 0.7 |
| 11a | 0.11 ± 0.03 | 0.59 ± 0.08 | 0.37 ± 0.05 | 0.38 ± 0.14 | 0.62 ± 0.07 |
| 14 | 0.025 ± 0.005 | 0.27 ± 0.03 | 0.23 ± 0.04 | 0.19 ± 0.02 | 0.26 ± 0.03 |

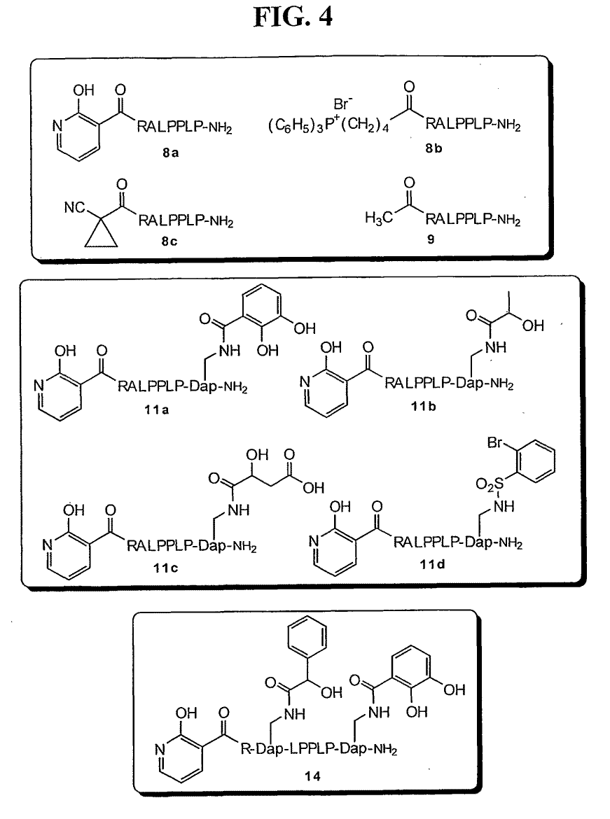

Our initial screen of the heptapeptide-based Library I revealed three lead derivatives. These leads were resynthesized on the Rink resin as the C-terminal amide-capped derivatives (compounds 8a-c; FIG. 4), purified, and their IC₅₀ values subsequently determined using the ELISA assay described in the previous paragraph. Their relative affinities ranged from 15 μM for the 2-hydroxynicotinic acid derivative 8a up to 45 μM for the cyclopropyl derivative 8e. K_{D} values were obtained via equilibrium dialysis using the fluorescent intensity inherent within the nicotinic acid moiety. The K_{D} for the lead derivative from Library I, compound 8a, is 1.2±0.2 μM, a nearly 15-fold improvement relative to the corresponding acetylated derivative 9 (K_{D}=15±4 μM). As expected, the parent peptide 9 is unable to discriminate between the various SH3 domains of the Src family members. By contrast, some selectivity in favor of Fyn is apparent with 8a, particularly with respect to the SH3 domains from Lck and Hck. However, compound 8a does not distinguish between the SH3 domains of Fyn, Yes, and Src. Although the molecular basis for this emerging selectivity remains to be resolved, we do note that, on the basis of sequence identity, Lck and Hck (Group B Src kinases) lie on a separate branch of the evolutionary tree from Fyn, Yes, and Src (Group A Src kinases) (Gu and Gu, 2003). This might account for the modest selectivity that compound 8a exhibits for the Fyn SH3 domain versus those from Lck and Hck.

With a biasing element at the N-terminus of Arg-Ala-Leu-Pro-Pro-Leu-Pro in place, we subsequently examined the effect of non-natural substituents at various internal sites of the consensus sequence peptide. Specifically, three sublibraries of the peptide 8a lead were prepared containing Dap replacements at Leu-3 (Library II), Leu-6 (Library III), and at the C-terminus (Library IV). These libraries were prepared as depicted in FIG. 2. In all three cases (10a-c), the Dap-containing 2-hydroxynicotinic acid-derivatized peptides were synthesized via an Fmoc protocol on the disulfide Tentagel resin. Following completion of the peptide framework, the Adpoc protecting group on the Dap side chain was selectively removed under mildly acidic conditions, the peptide-resin distributed to individual microwells, and the Dap amino group subsequently acylated with 720 different carboxylic acid derivatives (Libraries II-IV). As with Library I, each of the three sublibraries contained 720 distinct and physically separated members. The 2160 compounds that constitute Libraries II-IV were screened using the ELISA assay described above. The lead compounds from this screen were obtained exclusively from Library IV. These compounds were resynthesized on the Rink resin as the C-terminal amide-capped derivatives (11a-d, FIG. 4), purified, and subsequently assessed for Fyn SH3 affinity. Compound 11a exhibits a K_{D} of 110±30 nM, more than a 100-fold enhancement in affinity relative to the parent compound 9. In addition, a slight improvement in selectivity for the Fyn SH3 domain relative to Lck and Hck is apparent as is the emergence of discriminatory behavior versus that of Yes and Src.

Based on the optimized sequence contained in 11a, we prepared a second sublibrary containing molecular diversity introduced at the Ala-2 position. Library V contains two Dap residues, each of which must be selectively modified. A number of synthetic strategies are possible. However, given the possible reactivity of the catechol hydroxyl moieties of the Dap substituent in 11a, we decided to introduce the 2,3-dihydroxybenzoic acid moiety after completion of the peptide framework (FIG. 2). This required the use of two different Dap residues that were differentially side chain protected. The peptide-Tentagel resin 12 prepared via Fmoc chemistry. The Adpoc group on the C-terminal Dap residue was selectively removed with 1% CF₃CO₂H and 2,3-dihydroxybenzoic acid was subsequently coupled to the freed amino side chain (13). The Alloc protected Dap moiety at position 2 was then removed using Pd^{O}, and the resultant peptide-resin containing the free amine was distributed in equal amounts to 720 individual microwells for coupling with activated carboxylic acids (Library V). The peptide library was then side chain deprotected, extensively washed and subsequently released from the Tentagel resin using assay buffer. Screening of Library V furnished a single lead compound, which was resynthesized, purified, and evaluated as a ligand (14) for the SH3 domain of Fyn. As depicted in Table 1, peptide 14 displays a K_{D} of 25±5 nM for the SH3 domain of Fyn, a nearly 1,000-fold enhanced affinity relative to the starting parent peptide 9. In addition, the former exhibits a 10-fold selective affinity for Fyn versus SH3 domains from other Src kinase family members. To the best of our knowledge, this is the first example of a ligand that is able to distinguish between the SH3 domains of this closely related group of protein kinases.

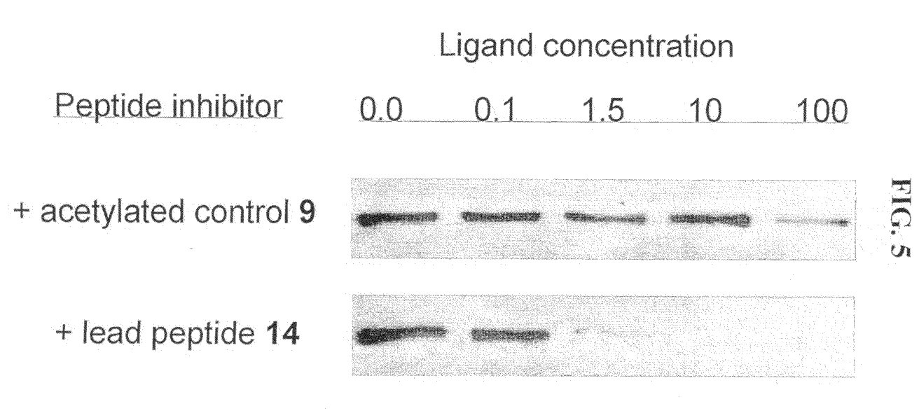

As noted in the Introduction, Fyn is known to play an important role in mediating the activation and proliferation of T cells via a signaling pathway that emanates from the T cell receptor. Recent studies have demonstrated that WASp is a participant in this pathway by directly interacting with and undergoing phosphorylation by Fyn (Banin et al., 1996; Badour et al., 2004). The association of a proline rich region on WASp with the SH3 domain of Fyn drives the formation of the transient WASp/Fyn complex. Fyn-mediated WASp phosphorylation at Tyr-291 releases WASp from an autoinhibitory conformation. Upon activation, WASP induces Arp2/3 activity, which in turn, promotes actin polymerization. In an apparently analogous fashion, the B cell lineage Src kinase, Hck, likewise interacts with WASp in an SH3 domain-dependent manner (Scott et al., 2002). For our initial studies, we examined the ability of compound 14 to block the interaction of WASp with the Fyn SH3 domain. We employed the human leukemia monocyte U937 cell line that was previously used to identify WASp as a Fyn SH3 domain interacting protein (Banin et al., 1996). Lysates from both undifferentiated U937 cells and phorbol 12-myristate 13-acetate-treated cells were employed (Schwende et al., 1996). Both protocols furnished similar results. The lysates were separately added to a mixture of glutathione-sepharose beads saturated with Fyn-SH3 GST fusion protein and varied amounts of the Fyn SH3 ligand 14 or the corresponding acetylated parent peptide 9. The sepharose beads were collected, the bound proteins released and fractionated by SDS-PAGE, and WASp visualized via chemiluminescence using an anti-WASp monoclonal antibody. As is evident from FIG. 5, ligand 14 disrupts formation of the WASp/Fyn complex with a greater than 100-fold enhanced potency relative to peptide 9.

### Conclusions

We have acquired a Fyn SH3 domain-selective ligand via an iterative library-based approach. This strategy relies upon the identification of widely distributed interaction sites on the surface of the target protein. Our results demonstrate that, even for a protein as small as an SH3 domain, potent ligands can be prepared using the stepwise protocol outlined in FIG. 1. To the best of our knowledge, ligand 14 displays the highest affinity ever reported for an SH3 domain from the Src kinase family. Furthermore, we have observed for the first time, the emergence of a ligand that distinguishes between the SH3 domains (˜80% sequence identity) of the highly conserved Src family of proteins. Clearly, it would be extremely useful to have an assortment of relatively low molecular weight ligands that selectively target individual members of the Src kinase family. These studies are in progress as is an examination of the molecular basis for the potency and selectivity associated with ligand 14.

### Significance

Signal transduction is primarily driven by protein-protein interactions. Given the important role that cell signaling plays in both normal and abnormal cellular events, there has been intense interest in the acquisition of inhibitory agents that block signaling pathways by interfering with the ability of proteins to recognize their appropriate binding partners. SH3 domains are amongst the smallest of all known protein binding modules and thus represent a significant challenge in terms of acquiring high affinity ligands. We have developed a straightforward stepwise strategy that has furnished an extremely potent ligand for the Fyn SH3 domain. The latter exhibits a sequence identity of 80% with other SH3 domains from the Src family of protein kinases. The Fyn ligand identified in this study represents the first example of a small non-protein molecule that is able to distinguish amongst the Src SH3 domain proteins.

### Experimental Procedures

The resins and reagents used for solid phase peptide synthesis, including Tentagel resin, Rink resin, N-9-fluorenylmethyloxycarbonyl (Fmoc)-L-amino acids, N,N,N′,N′-tetramethyl-(succinimido)uranium tetrafluoroborate (TSTU), benzotriazole-1-yloxytrispyrrolidinophosphonium hexafluorophosphate (PyBOP), 1-hydroxybenzotriazole (HOBt), were purchased from Advanced ChemTech. Peptide synthesis grade dichloromethane (DCM), N,N-diisopropylethylamine (DIPEA), isopropyl alcohol (IPA), dimethylformamide (DMF) and trifluoroacetic acid (TFA) were purchased from Fisher and piperidine was obtained from Lancaster. 2-Fmoc-3-[1-(1′-Adamantyl)-1-methyl-ethoxycarbonyl]-diaminopropionic acid (Fmoc-Dap(Adpoc)-OH) was obtained from Bachem. Triisopropylsilane (TIS) was purchased from Acros. The 720 carboxylic acids used for the preparation of the peptide libraries were purchased from Aldrich. The GST-SH3 fusion proteins, Fyn (85-139) and Lck (54-120), and polyclonal rabbit anti-GST HRP conjugate antibody were purchased from Santa Cruz Biotechnology. Peroxidase substrate (1-step Turbo trimethylbenzidine ELISA), streptavidin-coated 96-well plates, and Slide-A-Lyzer dialysis slide cassettes (Mᵣ 10,000 cutoff) were purchased from Pierce. Solvent resistant MultiScreen 96-well filter plates and the MultiScreen 96-well filter plate vacuum manifold were purchased from Millipore Corp.

One dimensional and two dimensional ¹H NMR spectra of the peptide inhibitors were recorded on a DRx600 MHz Spectrometer in H₂O/D₂O (90:10), and chemical shifts are reported in parts per million (ppm) downfield from (CH₃)₄Si. The molecular weights of the peptides were analyzed with MALDI mass spectrometry on an Applied Biosystems Voyager DE STR and ESI-MS on an Applied Biosystems MDS SCIEX API Qstar Pulsar I. Reverse phase high performance liquid chromatography (RP-HPLC) was performed on a Waters SD-200 solvent delivery system equipped with a 500 UVis-absorbance detector and recorded on an Apple Macintosh computer using model 600 software (Applied Biosystems Inc.). Chromatographic separations were achieved using linear gradients of buffer B in A (A=0.1% aqueous TFA; B=0.1% TFA in CH₃CN) over 50 min at a flow rate of 12 mL/min using a detection wavelength of 218 nm on Delta-Pak C18 (300 Å, 15 μm, 3×15 cm) column.

Peptide Synthesis. Peptides were synthesized using a standard Fmoc solid phase peptide synthesis (SPPS) protocol on an Innova 2000 platform shaker, or with an Advanced Chemtech Model 90 Tabletop Peptide Synthesizer.

Synthesis of Library I. 5 g of Tentagel S COOH (90 μm, 0.2 mmol/g) and 1.94 g (15 mmol) of DIPEA were successively added to a solution of 1.5 g (5 mmol) of TSTU in 20 mL of DMF. The mixture was shaken for 2 hr at ambient temperature. Subsequently, a mixture of 2.25 g (10 mmol) of cystamine dihydrochloride and 2.02 g (20 mmol) of N-methylmorpholine (NMM) in 20 mL of water was slowly added to the Tentagel reaction mixture. Heat was evolved upon addition. Upon cooling to room temperature, the reaction vessel was sealed and shaken overnight. The resin was then drained and washed successively with H₂O (3×20 mL), DMF (3×20 mL), and CH₂Cl₂ (3×20 mL). The free amine substitution level on linker-coupled resin was found to be 0.05 mmol/g. The linker-coupled resin (5 g) was successively submitted to coupling reactions with the required amino acids followed by removal of the Fmoc protecting group via standard conditions (vide infra). Each residue was coupled for 3 hr, and coupling efficiencies were determined by quantitative ninhydrin reaction. The standard coupling conditions employed 5 equiv. of Fmoc-amino acid, 5 equiv. of HOBt, 5 equiv. of PyBOP and 10 equiv. of NMM in 30 mL DMF with shaking for 3 hr. After each coupling step, the resin was successively washed with DMF (3×20 mL), isopropyl alcohol (3×20 mL), and CH₂Cl₂ (3×20 mL). The Fmoc protecting group was removed with 20% piperidine in DMF (shaking for 20 min). After the assembly of the consensus sequence of Fmoc-Arg(Pbf)-Ala-Leu-Pro-Pro-Leu-Pro-S—S-Tentagel-Resin, the Fmoc group at the amino terminus was removed and the resin extensively washed and subsequently dried in vacuo. The peptide-bound resin was distributed in 5 mg quantities into individual wells of solvent-resistant MultiScreen™ 96-well filter plates-(8 plates total). To each well was added a solution of a carboxylic acid (200 equiv.) in 100 μL DMF and a second solution containing PyBOP (200 equiv.), HOBt (200 equiv.), and NMM (400 equiv.) in 100 μL of DMF. A total of 720 different carboxylic acids were employed. The plates were gently shaken overnight, and then each well subjected to a series of washing steps (3×200 μL of DMF, 3×200 μL of isopropyl alcohol, and 3×200 μL of CH₂Cl₂). The -side chain protecting group Pbf was removed via treatment with TFA:H₂O:TIS (v/v 95:2.5:2.5) for 2×3 hr at ambient temperature. The resin was washed with DMF (3×20 mL), isopropyl alcohol (3×20 mL), and CH₂Cl₂ (3×20 mL) and the peptide-nonpeptide conjugates subsequently cleaved from the disulfide-containing resin with 10 mM dithiothreitol (DTT) in 50 mM Tris, pH 7.5 (1×200 μL for 3 hr and 2×150 μL for 3 hr each) and filtered into a receiving set of 96-well plates using a vacuum manifold (final volume of 500 μL). The coupling efficiency of the acylation reaction and the purity of peptide-nonpeptide conjugates were assessed via the ninhydrin test and RP-HPLC, respectively. No free N-terminal peptide was detected, and >90% of total ligand was cleaved from the resin with the first DTT cleaving step. The final two DTT washings removed the residual resin-bound peptide. Compound purity was >90% as assessed by HPLC, and the HPLC-purified compounds (i.e. removal of Tris buffer and DTT) were characterized by MALDI-MS. These peptides, containing 720 different groups at the N-terminus of consensus sequence in 8 plates, comprise Library I.

Synthesis of Libraries II, III and IV. Construction of Libraries II, III and IV is depicted in FIG. 2. The side chain protected consensus sequence Fmoc-Arg(Pbf)-Ala-Leu-Pro-Pro-Leu-Pro-S—S-Tentagel-Resin was assembled on the Tentagel resin as described in the previous paragraph with the following substitutions: the N-terminus leucine was replaced with a Dap(Adpoc)-OH to afford Library II, the C-terminus leucine was replaced with a Dap(Adpoc)-OH to afford Library III, a Dap(Adpoc)-OH residue was inserted into the C-terminus of the sequence to afford Library IV. The N-terminus Fmoc group (5 g resin) was then removed with 20% piperidine in DMF and the resin was thoroughly washed with DMF, IPA and CH₂Cl₂. A solution containing 2-hydroxynicotinic acid (695.5 mg, 8 equiv.), PyBOP (2.6g, 8 equiv.), HOBt (765 mg, 8 equiv.), NMM (1.26 g, 20 equiv.) in 30 mL DMF were added and the slurry was shaken overnight at room temperature. The solvent was removed from the resin and the resin subsequently washed with DMF (3×20 mL), isopropyl alcohol (3×20 mL), and CH₂Cl₂ (3×20 mL). The Adpoc group in these three sequences (5 g resin) was selectively removed with 40 mL of 3% TFA in CH₂Cl₂ (3×5 min) and the resin bearing free amine on Dap residue washed, dried, and then added in 5 mg quantities to the individual wells of 8 solvent-resistant MultiScreen™ 96-well filter plates. The following procedures as described for Library I, were employed: the resin in each well was coupled with one of 720 different carboxylic acids, the side chain protecting groups were removed, and the peptides were cleaved from the resin to furnish Library II, III and IV.

Synthesis of Library V. Construction of Library V was depicted in FIG. 2. The side chain protected consensus sequence Fmoc-Arg(Pbf)-Dap(Alloc)-Leu-Pro-Pro-Leu-Pro-Dap(Adpoc)-S—S-Tentagel-Resin was assembled on the Tentagel resin as previously described. The N-terminus Fmoc group (5 g resin)-was removed with 20% piperidine in DMF and the resin was washed and coupled to 2-hydroxynicotinic acid in the same fashion described in the previous paragraph. The Adpoc group in the sequence (5 g resin) was selectively removed with 40 mL of 3% TFA in CH₂Cl₂ (3×5 min) and the resulting free amine on the side chain of the Dap residue was coupled with 770 mg (5 mmol) of 2,3-dihydroxybenzoic acid in the presence of 1.9 g (5 mmol) of HATU, 0.765 g (5 mmol) of HOAt, and 1.29 g (10 mmol) of DIPEA in 40 mL of DMF. The reaction mixture was shaken overnight at room temperature. After the solvent was removed and the resin subsequently washed, a solution of 58 mg (0.05 mmol, 0.2 equiv.) of Pd(PPh₃)₄ in 30 mL solvent of THF:DMSO:(0.5 M HCl):morpholine (v/v 20:20:10:1) was added to the resin and shaken for 24 hr at room temperature (Morley, 2000), to selectively deprotect the Alloc group protecting the N-terminus Dap residue. The doubly modified peptide-resin of (2-hydroxynicotinic)-HN-Arg(Pbf)-Dap-(NH₂)-Leu-Pro-Pro-Leu-Pro-Dap-(NH-2,3-dihydroxybenzoic)-Phe-S—S-Tentagel-Resin at this stage was thoroughly washed, dried in vacuum, and added in 5 mg quantities to the individual wells of 8 solvent-resistant MultiScreen™ 96-well filter plates. The resin in each well was then coupled with one of 720 different carboxylic acids, the side chain protecting groups were removed, and the peptides were cleaved from the resin to furnish Library V.

Peptide 8a: The peptide resin Fmoc-Arg(Pbf)-Ala-Leu-Pro-Pro-Leu-Pro-NH-Rink-resin was prepared using the protocol described above for Library I using the Rink SS resin instead of TentaGel S COOH. After the Fmoc group was removed on the N-terminus and the resin was thoroughly washed, a solution containing 2-hydroxynicotinic acid (5 equiv.), PyBOP (5 equiv.), HOBt (5 equiv.), NMM (15 equiv.) in DMF was added and the slurry shaken overnight at room temperature to afford the peptide modified at the N-terminus. The peptide was subsequently deprotected and released from the resin in one step using a TFA/TIS/H₂O (v/v 95:2.5:2.5) cocktail for 2-3 hr. ESIMS m/z calculated for C₄₂H₆₅N₁₁O₁₀ 884.03 (MH⁺), Found m/z 884.8

Peptide 11a: The peptide resin (2-hydroxynicotinic-NH-Arg(Pbf)-Ala-Leu-Pro-Pro-Leu-Pro-Dap(Adpoc)-NH-Rink-resin was prepared using the protocol described above for Library IV using Rink SS resin instead of TentaGel S COOH. After Adpoc deprotection with 3% TFA in DCM and resin washing, a solution of 2,3-dihydroxybenzoic acid (5 equiv.), HATU (5 equiv.), HOAt(5 equiv.), DIPEA (10 equiv.) in DMF was added and the slurry shaken for 3 hr at room temperature to afford the peptide modified at the C-terminus. The peptide was subsequently, deprotected and released from the resin in one step using a TFA/TIS/H₂O (v/v 95:2.5:2.5) cocktail for 2-3 hr. ¹HNMR(600 MHz, H₂O, ppm) 7.81 (1H), 6.75 (1H), 8.49 (1H) for N-terminus nicotinic acid aromatic hydrogens; 10.31 (1H, s, CONH), 4.55 (1H, C_{α}H), 1.95 (1H, C_{β}H), 1.88 (1H, C_{β}H), 1.69 (2H, C_{γ}H₂), 3.23 (2H, C_{δ}H₂), 7.24 (N^{ε}H) for Arg-1; 8.58 (1H, s, CONH), 4.37 (1H, C_{α}H), 1.38 (2H, C_{β}H₂) for Ala-2; 8.31 (1H, s, CONH), 4.62 (1H, C_{α}H), 1.56 (2H, C_{β}H₂), 1.67 (2H, C_{γ}H₂), 0.91 (2H, C_{δ}H₂) for Leu-3; 4.70 (1H, C_{α}H), 2.34 (1H, C_{β}H), 1.90 (1H, C_{β}R), 2.03 (2H, C_{γ}H2), 3.83 (1H, C_{δ}H), 3.67 (1H, C_{δ}) for Pro-4; 4.42 (1H, C_{α}H), 2.27 (1H, C_{β}H), 1.90 (1H, CH), 2.03 (2H, C_{γ}H₂), 3.79 (1H, C_{δ}H), 3.65 (1H, C_{δ}H) for Pro-5; 8.16 (1H, s, CONH), 4.57 (1H, C_{α}H), 1.45 (2H, C_{β}H₂), 1.65 (2H, C_{γ}H₂), 0.86 (2H, C_{δ}H₂) for Leu-6; 4.39 (1H, C_{α}H), 2.27 (1H, C_{β}H), 1.90 (1H, C_{β}H), 2.03 (2H, C_{γ}H₂), 3.84 (1H, C_{δ}H), 3.66 (1H, C_{δ}H) for Pro-7; 8.34 (1H, s, CONH), 4.66 (1H, C_{α}H), 3.86 (1H, C_{β}H), 3.77 (1H, C_{β}H), 7.61 (amide NH of C-terminus), 7.25 (amide NH of C-terminus) for Dap-8; 8.74 (1H, s, CONH); 7.10 (1H), 6.88 (1H), 7.25 (1H) for C-terminus 2,3-dihydroxyl benzoic acid. ESIMS m/z calculated for C₅₂H₇₆N₁₄O₁₃ 1105.25 (MH⁺), Found m/z 1104.6.

Peptide 14: The peptide resin (2-hydroxynicotinic-NH)-Arg(Pbf)-Dap(alloc)-Leu-Pro-Pro-Leu-Pro-Dap(NH-2,3-dihydroxybenzoic)-NH-Rink-resin was prepared using the protocol described above for Library V using Rink SS resin. After alloc deprotection with Pd(0) and resin washing, a solution of (R)-(−)-Mandelic acid (5 equiv.), PyBOP (5 equiv.), HOBt (5 equiv.), NMM (15 equiv.) in DMF was added and the slurry was shaken for 3 hr at room temperature to afford the second Dap residue modification. The peptide was subsequently released from the resin in one step using a TFA/TIS/H₂O (v/v 95:2.5:2.5) cocktail for 2-3 hr. ¹H NMR (600 MHz, H₂O, ppm) 7.78 (1H), 6.70 (1H), 8.32 (1H) for N-terminus nicotinic acid aromatic hydrogens; 10.30 (1H, s, CONH), 4.48 (1H, C_{α}H), 1.88 (1H, C_{β}H), 1.80 (1H, C_{γ}H), 1.65 (2H, C_{γ}H₂), 3.17 (2H, C_{δ}H₂), 7.19 (N^{ε}H) for Arg-1; 8.57 (1H, s, CONH), 4.60 (1H, C_{α}H), 3.82 (1H, C_{β}H), 3.57 (1H, C_{β}H) 8.49 (CONH, s, CONH of Dap-2-hydroxyl benzoic acid) for Dap-2; 8.40 (1H, s, CONH), 4.62 (1H, C_{α}H), 1.59 (2H, C_{β}H₂), 1.64 (2H, C_{γ}H₂), 0.90 (2H, C_{δ}H₂) for Leu-3; 4.65 (1H, C_{α}H), 2.32 (1H, C_{β}H), 1.89 (1H, C_{β}H), 2.04 (2H, C_{γ}H₂), 3.86 (1H, C_{δ}H), 3.62 (1H, C_{δ}H) for Pro-4; 4.42 (1H, C_{α}H), 2.27 (1H, C_{βH),} 1.89 (1H, C_{β}H), 2.0 (2H, C_{γ}H₂), 3.73 (1H, C_{δ}H), 3.60 (1H, C_{δ}H) for Pro-5; 8.15 (1H, s, CONH), 4.57 (1H, C_{α}H), 1.45 (2H, C_{β}H₂), 1.64 (2H, C_{γ}H₂), 0.85 (2H, C_{δ}H₂) for Leu-6; 4.42 (1H, C_{α}H), 2.27 (1H, C_{β}H), 1.89 (1H, C_{β}H), 2.0 (2H, C_{γ}H₂), 3.82 (1H, C_{δ}H), 3.65 (1H, C_{δ}H) for Pro-7; 8.34 (1H, s, CONH), 4.64 (1H, C_{α}H), 3.86 (1H, C_{β}H), 3.76 (1H, C_{β}H), for Dap-8; 8.73 (1H, s, CONH); 7.09 (1H), 6.87 (1H), 7.22 (1H) for C-terminus 2,3-dihydroxyl benzoic acid. ESIMS m/z calculated for C₆₀H₈₂N₁₄O₁₆ 1255.38 (MH⁺), Found m/z 1255.9.

Synthesis of peptoid ligands 5-7: Peptoid ligands 5-7 were synthesized on the Rink resin using a standard solid phase peptide synthesis protocol with the following exception to introduce the N-glycine-substituted peptoid moiety: after the assembly of the Pro-Arg-Asn-Arg-Pro-Arg-Ala sequence on the Rink resin, the N-terminus of the peptide was acylated by reaction with equal volumes of 1 M bromoacetic acid in DCM (10 equiv.) and 1 M diisocarbodiimide in DMF (10 equiv.) twice for 1 hr each; nucleophilic displacement was effected with 2 M N-(S)-phenylethyl(Nspe) amine in dimethyl sulfoxide (15 molar equiv.) twice for 2 hr each. The subsequent Fmoc amino acid (10 equiv.) was appended by using the coupling reagent HATU (10 equiv.) and DIPEA (18 equiv., twice for 2 hr each). Two Fmoc-Aminohexanoic acid (Fmoc-Ahx) residues were coupled to the N-terminus as spacers between the peptoid ligand and the appended moiety (biotin, 6; fluorescein, 7). Biotinylation was effected on the resin with 4 equiv. of biotin in DMSO/DMF (v/v 50:50) in the presence of coupling reagents (PyBOP, 4 equiv.; HOBt, 4 equiv.; NMM, 4 equiv.) at room temperature for 3 hr. FITC labeling was likewise conducted on the resin with 2 equiv. of FITC dissolved in pyridine/DMF/DCM (v/v 12:7:5) at room temperature in the dark overnight. Peptoid ligands 5-7 were subsequently deprotected and released from the Rink resin using a TFA/TIS/H₂O cocktail (v/v 95:2.5:2.5) for 3 hr. ESIMS of 6 calculated for C₉₆H₁₅₀N₂₆O₂₀S m/z 2020.45 (MH⁺), Found m/z 2020.78. ESIMS of 7 calculated for C₁₀₇H₁₄₇N₂₅O₂₃S m/z 2183.53 (MH⁺), Found m/z 2184.13.

Screening of the Peptide/Nonpeptide Conjugate Library. An ELISA assay was employed to screen the library for SH3 affinity. 100 μL of biotinyl-6-aminocaproyl-YAPPL-x(Nspe)-PRNRPRA 6 (333 ng/mL in 50 mM Tris, 150 mM NaCl, pH 7.5) was added to each well of streptavidin-coated 96-well microtiter plates. The plates were shaken overnight at 4° C. and rinsed with TBS (50 mM Tris, 150 mM NaCl, pH 7.5, 3×200 μL). Each well was then blocked with 100 μL of a solution containing 2% BSA and 0.2% Tween 20 in TBS (2 hr at RT). The wells were rinsed with 4×200 μL of a standard “BSA-T-TBS” solution (0.2% BSA, 0.1% Tween 20, TBS). A 50 μL solution of the peptide/nonpeptide conjugate (100 nM, in BSA-T-TBS) from the library and a 50 μL solution of the Fyn SH3-GST fusion protein (32 ng/mL, in BSA-T-TBS) were added in each well and the plate was shaken for 2.5 hr at room temperature. The solutions were removed and each well rinsed with 4×200 μL BSA-T-TBS. 100 μL of horseradish peroxidase-conjugated rabbit polyclonal anti-GST antibody (100 ng/mL in BSA-T-TBS) was then added to each well and incubated for 2 hr at room temperature. After a series of final wash steps (4×200 μL BSA-T-TBS; 4×250 μL TBS), 100 μL of peroxidase substrate (1-step Turbo TMB-ELISA, trimethylbenzidine) was added to each well and incubated for 5-15 min. 100 μL of 1M sulfuric acid solution was introduced to stop the peroxidase reaction and absorbance was measured at 450 nm with a plate reader. IC₅₀ values were determined using the ELISA screening method around a 200-fold range of ligand concentrations.

WASP-Fyn binding assay. U937 cells were maintained in RPMI 1640 medium supplemented with 5% fetal bovine serum. To induce differentiation toward a monocytic lineage, exponentially growing U937 cells were sub-cultured at a density of 5×10⁵ cells ml⁻¹ and treated with PMA (Sigma) at a final concentration of 10 ng ml⁻¹ for 24 h. Cells were then harvested and washed in ice-cold phosphate-buffer saline, and lysed in 10 mM Tris-HCl, (pH 7.5), 1% (v/v) Triton X-100, 150 mM NaCl, 5 mM EDTA, 50 mM NaF, 1 mM phenylmethylsulfonyl chloride, 5 mM benzamidine, 1 mM NaVO₄ and protease inhibitors (5 mg/mL aprotinin, 5 mg/mL leupeptin, 5 mg/mL pepstatin). Protein concentrations were measured using the Bradford assay after preclearing the lysate with glutathione-sepharose beads. Aliquots of cell lysate were added to 20 μL of glutathione-sepharose beads saturated with Fyn-SH3 GST fusion protein in presence of peptide 14 or acetylated control 9. Beads and lysate were gently shaken for 2 h at 4° C. and then washed 5 times with lysis buffer. Bound proteins were eluted into gel loading buffer by boiling, fractionated by SDS-PAGE under reducing conditions, and then electrotransferred to polyvinylidene difluoride (PVDF) membranes (Millipore). The membranes were blocked via overnight exposure to PBS buffer with 5% non-fat milk. The amount of WASp associated with Fyn-SH3 was detected by sequential incubation with anti-WASp monoclonal antibody (Pharmingen, Calif.), secondary HRP conjugated antibody (Pharmingen, Calif.), and then visualized using an enhanced chemiluminescent (ECL) western blotting reagent according to the manufacturer's instructions (Amersham). The same PVDF membrane was stripped in 0.2 N NaOH solution for 10 min, and blotted against anti GST HRP antibody (Santa Cruz Biotech, CA) to verify that an equal amount of Fyn-SH3 protein was loaded into each lane.

Determination of K_{d} Values. Peptides containing a nicotinic acid substituent at their N-terminus are highly fluorescent and exhibit little or no change fluorescence upon coordination to the Fyn SH3 domain. Therefore, the K_{D} values for the SH3 complexes of these species were determined via equilibrium dialysis. All samples were prepared in buffer containing TBS and 1 mM DTT at pH 7.5. Slide-A-Lyzer dialysis slide cassette (0.1-0.5 mL capacity) was employed and contained 30 nM or 500 nM Fyn SH3-GST fusion proteins. The cassettes (400 μL final volume) were placed in a beaker containing a volume of buffer solution (TBS and 1 mM DTT at pH 7.5) that was at least 400-fold greater than that of the sample volume in the dialysis cassette. As a consequence, concentrations of non-SH3-bound peptide were held constant in the dialysis slide cassette over the course of the experiment. Equilibrium dialysis experiments were performed over a period of at least 16 hr and maintained at 4°C. Differences in the fluorescence between the solution in the slide cassette and that in the beaker were measured. The excitation wavelength employed for the peptides were 328 nm. Emission was monitored at 378 nm. The K_{d} values were calculated from the following equation for fluorescent peptides 8a, 11a, 14 and 7, where [E]ₜ=total enzyme concentration; [E·L]=enzyme-peptide complex; [L]=free peptide concentration.

$${K}_{d} = \frac{\{ {[ E ]}_{t} - [ E \cdot L ] \} [ L ]}{[ E \cdot L ]}$$

For non-fluorescent peptide 9, the following equation was used for the determination of K_{d}′:

$${K}_{d}^{′} = \frac{{K}_{d} \cdot [ {L}^{′} ]}{\frac{{[ E ]}_{t} \cdot [ L ]}{[ E \cdot L ]} \cdot ( {K}_{d} + [ L ] )}$$

Where K_{d}=the dissociation constant of competition fluorescent peptide, [L′]=total lion-fluorescent peptide concentration, [L]=total competitive fluorescent peptide concentration, [E]ₜ=total SH3 domain concentration; [E·L]=enzyme-peptide (fluorescent competitive peptide) complex concentration.

In view of the above, it will be seen that the several advantages of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

1. A compound comprising A-Arg-B-Leu-Pro-Pro-Leu-Pro-C, wherein A=moiety I, II, or III 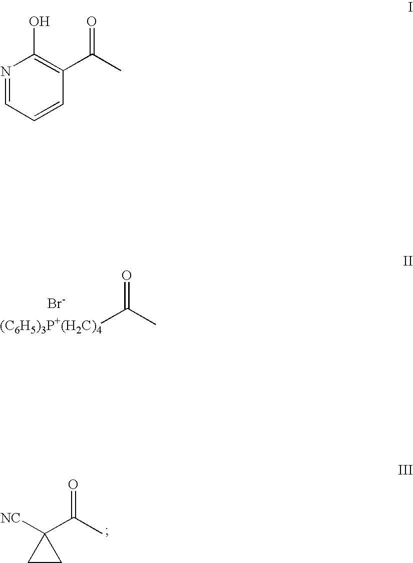
B=Ala, (L)-2,3-diaminopropionic acid (Dap) or 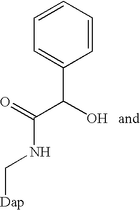
C=any moiety,
wherein any amino acid can alternatively be an analogous peptidomimetic.

2. The compound of claim 1, wherein C is NH₂, an organic compound less than 500 Dalton or an organic compound less than 500 Dalton and a resin.

3. The compound of claim 1, wherein C is NH₂,

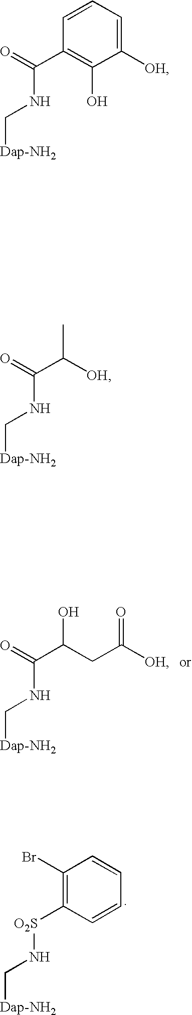

4. The compound of claim 1, wherein C is

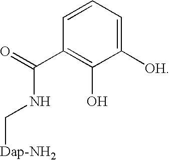

5. The compound of claim 1, wherein A is moiety I.

6. The compound of claim 1, wherein A is moiety I and C is

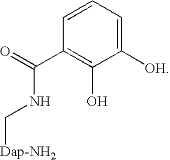

7. The compound of claim 6, wherein B is

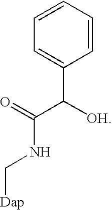

8. The compound of claim 1, comprising

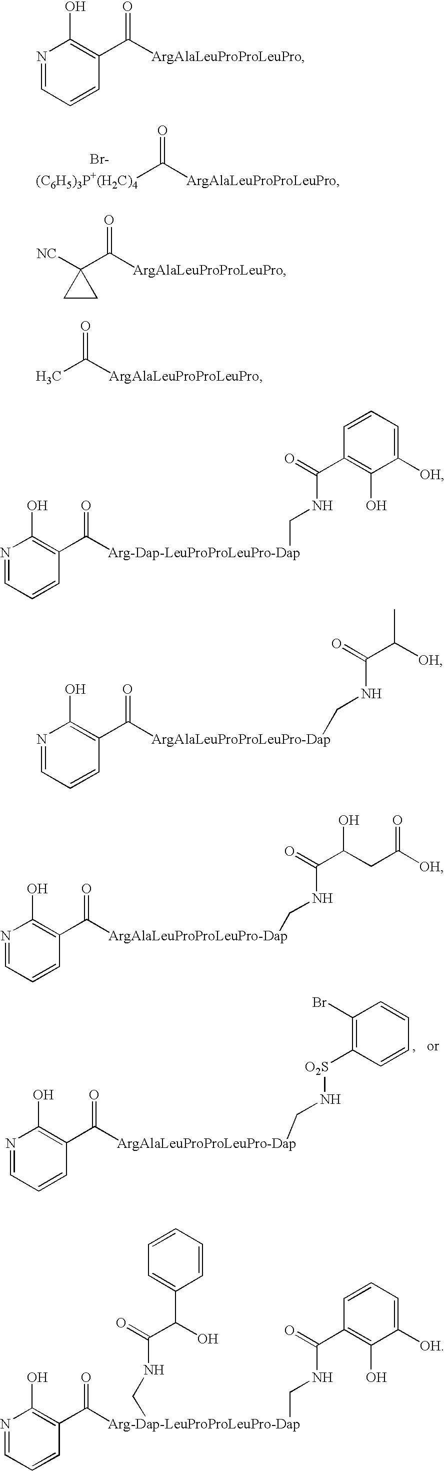

9. The compound of claim 1, consisting of

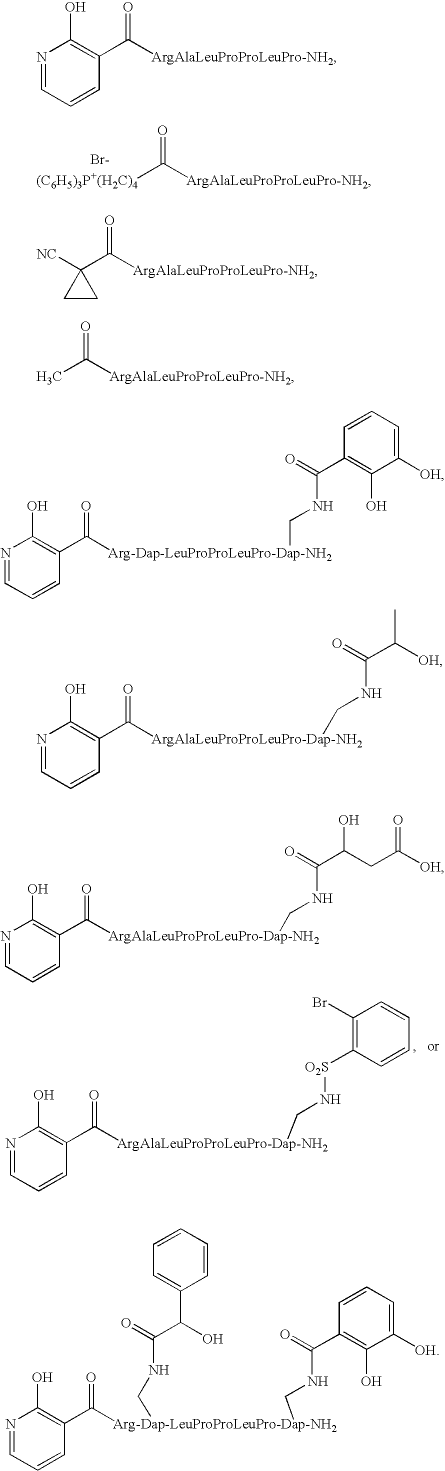

10. The compound of claim 1, comprising

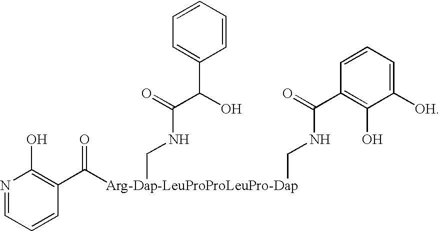

11. The compound of claim 1, wherein the compound is

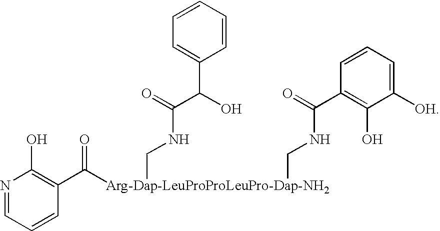

12-31. (canceled)

32. A composition comprising the compound of claim 1 in a pharmaceutically acceptable carrier.

33-44. (canceled)

45. The composition of claim 32, formulated in unit dosage for treatment of a deleterious condition in a mammal.

46-49. (canceled)

50. A combinatorial library comprising a plurality of compounds, each compound comprising A-Arg-B-Leu-Pro-Pro-Leu-Pro-C, wherein
A is H, NH₂ or an organic compound less than about 500 Dalton,
B is Ala, Dap, or Dap-D, where D is an organic compound less than about 500 Dalton, and
C is any moiety, and wherein wherein any amino acid can alternatively be an analogous peptidomimetic,
wherein each compound is different.

51-65. (canceled)

66. A method of identifying a compound that binds to an SH3 domain, the method comprising
creating a first combinatorial library of claim 50;
screening the compounds in the first combinatorial library for binding to the SH3 domain; and
identifying any compounds in the first combinatorial library that bind to the SH3 domain.

67-70. (canceled)

71. A method of inhibiting an activity of a protein comprising an SH3 domain, the method comprising identifying a compound that inhibits the SH3 domain by the method of claim 66, then contacting the compound with the protein in a manner sufficient to inhibit the activity of the protein.

72-84. (canceled)

85. A method of inhibiting the activity of a protein comprising an SH3 domain, the method comprising combining the protein with the compound of claim 11 in a manner sufficient to inhibit the activity of the protein.

86-102. (canceled)

103. A method of treating a mammal having a deleterious condition that is mediated by a protein comprising an SH3 domain, the method comprising administering the composition of claim 32 to the mammal.

104-112. (canceled)
